(19) United States
(12) Patent Application Publication
(10) Pub. No.: US 2012/0064477 A1
(43) Pub. Date: Mar. 15, 2012

(54) IMPRESSION TRAY, AND METHOD FOR CAPTURING STRUCTURES, ARRANGEMENTS OR SHAPES, IN PARTICULAR IN THE MOUTH OR HUMAN BODY

(75) Inventor: Fritz SCHMITT, Wasserbillig (LU)

(73) Assignee: Medentic S.A., Wasserbilling (LU)

(21) Appl. No.: 13/184,441

(22) Filed: Jul. 15, 2011

Related U.S. Application Data
(63) Continuation of application No. PCT/EP2009/006474, filed on Sep. 7, 2009.

(30) Foreign Application Priority Data
Jan. 15, 2009 (DE) 20 2009 000 458.9
Apr. 23, 2009 (IB) PCT/IB2009/006054
Jul. 28, 2009 (EP) EP 09166523

(51) Int. Cl. A61C 9/00; A61B 1/24; A61B 6/14

(52) U.S. Cl. 433/29; 433/44; 433/215

(57) ABSTRACT

The invention relates to an impression tray, such as in particular a dental impression tray, which carries a deformable impression mass in order to prepare an impression of arrangements, shapes and/or dimensions, in particular in or on the human body, preferably in the mouth, and further preferred an impression of at least part of a tooth or of dental structures, wherein furthermore sensor devices are present, by means of which a change of at least one physical property and/or variable of the impression mass can be captured in a spatially resolved manner when preparing an impression and can be provided in a form that is suited for electronic data processing. The invention further relates to a method for capturing structures, arrangements or shapes, such as preferably for capturing dental structures, arrangements or shapes in the mouth or in the human body, whereby a deformable impression compound is brought onto or into the structures, arrangements or shapes in particular, is introduced, into the mouth or body and a change of at least one physical property and/or variable of the impression compound is transmitted there in a spatially resolved manner directly to sensor devices when preparing an impression and is captured by the sensor devices and, furthermore, provided in a form that is suitable for electronic data processing.

### CROSS REFERENCES TO RELATED APPLICATIONS

This application is a continuation of pending international patent application PCT/EP2009/006474, filed on Sep. 7, 2009 designating the U.S., which international patent application has been published in German language and claims priority from German utility model DE 20 2009 000 458.9, filed on Jan. 15, 2009, International patent application PCT/IB2009/006054 filed Apr. 23, 2009 and European patent application EP 09 166 523, filed Jul. 28, 2009. The entire contents of these prior applications are incorporated herein by this reference.

### BACKGROUND OF THE INVENTION

The invention relates to impression trays, such as in particular dental impression trays, and methods for capturing structures, arrangements or shapes, such as preferably for capturing dental structures, arrangements or shapes in the mouth or in the human body. Even though a significant application area for the invention lies in odontology, for capturing dental structures in the mouth of the patient, the invention can also be used in other areas of medicine, such as for example for determining arrangements and shapes as well as dimensions of surfaces (for example the arm, etc.) or bones, in order to prefabricate splints, prostheses or other aids, such as for example bone plates. Apart from the requirements for extreme accuracy, important aspects in the use of prostheses and aids are the production time up until when corresponding parts are available for use on or in the patient, easy handling by the doctor carrying out the capture and the fitting, and the stress on the patient from capturing the shape to making the fitting. Specifically, but not only, in odontology, the costs of the entire procedure, from the first appointment to obtain a diagnosis to the completion of the treatment, are also of immense importance.

For example in odontology, it is still customary to use impressions of the actual teeth, and plaster models prepared therefrom by manual work, in order to make the required prostheses, again by manual work. Not only does the overall procedure take a long time and cause correspondingly high costs, but also the accuracy is limited and often unsatisfactory, which in turn necessitates again laborious and expensive reworking.

So, in the meantime, methods whereby dental data and/or dental structures can be determined in the mouth of the patient in a computer-aided manner have become known, for example at trade fairs. However, these methods and technologies known from practice, referred to as computer-aided odontology, for capturing dental structures in the mouth of a patient have not been able so far to establish themselves significantly in the treatment of patients. This is due to the associated disadvantages.

In the case of one approach to this method and technology known from practice, first an impression of teeth or dental structures in the mouth of a patient is taken with a conventional elastic impression compound in what is known as a dental impression tray. This impression is then used to prepare a plaster model, which is scanned mechanically, optically or in some other way in order to obtain 3D data of the teeth or dental structures of a patient. By means of these 3D data, prostheses can then be produced in an automated process. This allows greater accuracy, comparatively rapid production and easy reworking or renewed production to be achieved. There are, however, still a series of disadvantages:
the preparation of a plaster model is outdated and still labor-intensive, since it can scarcely be automated, so the costs are still quite high,
this negative impression must be used to make a plaster model, the accuracy of which is compromised by the impression and itself again determines the accuracy of the later prosthesis,
the waiting time while the impression is taken is still unpleasant for the patient, since the elastic impression material has to be cured from a kneadable state (irreversible deformation) into an only elastically deformable state (reversible deformation),
once it has been used, the impression compound is “lost”, since it has indeed been cured into an only elastically deformable state (reversible deformation) and, moreover, must go to the laboratory as a mold for the plaster model, this material consumption also causing an environmental impact in the disposal of the impressions,
the transport of the impression to the laboratory takes time and entails the risk of the impression being damaged or even lost,
the impression compound to be used must be kept in sufficient quantities at dental surgeries, it also being possible for it to dry out if stored for too long,
when preparing the digital data from the plaster model, the latter or even the impression must be taken as a basis, without any possibility of referring back to the patient, so that anomalies can only be clarified laboriously by the dentist taking another impression from the patient, while prior/subsequent consideration of the area around neighboring teeth, for example, is no longer possible at all because of the further treatments that have normally already taken place (for example grinding down a tooth to a stump for fitting a crown), and
there is no possibility of further processing for production in computer-aided areas (digital data).

In order in turn to counter these disadvantages, optical video or scanner systems that are used directly in the mouth of a patient have been developed. Although they have become known for example at trade fairs, these systems have also not yet been able to break through into treatment in practice. The reason for this is that, although such systems theoretically allow some of the disadvantages of the technology and procedure of obtaining data of plaster models to be overcome, it is necessary in turn to accept other disadvantages that are inherent in the systems:

handling is very difficult, since with hand-held devices that have to be introduced into the patient's mouth there is no possibility, for example, of correctly capturing entire arches of teeth,

floor-mounted devices require patients to accept great discomfort, since the patient must indeed adapt to the requirements of such devices, such as for example constantly maintain certain positions of the head and jaw while images are recorded,

only optical 2D images can be taken with reasonable effort; mechanical scanning operations could only be performed with immense demands in terms of time,

personnel must be laboriously trained, and “clumsy” errors are difficult to eliminate,

there are glaring inaccuracies caused by saliva or shadow, and also the problem of missing or inaccurate references in images and scans taken, and further processing increases the expense, and

distortions caused by equipment defects, such as for example deposits on sensors/lenses, and personnel shortcomings or lack of concentration sometimes go unnoticed and lead to unusable results, which in some cases can only be noticed after a prosthesis has been produced.

### SUMMARY OF THE INVENTION

It is therefore one object of the invention to provide a device and method for capturing three-dimensional structures that overcome the disadvantages of the prior art, or at least reduce them.

For this purpose, the invention provides an impression tray, such as in particular a dental impression tray, which carries a deformable impression compound in order to prepare an impression of arrangements, shapes and/or dimensions, in particular in or on the human body, preferably in the mouth, and further preferred an impression of at least part of a tooth or of dental structures, wherein furthermore there are sensor devices, by means of which a change of at least one physical property and/or variable of the impression compound can be captured in a spatially resolved manner when preparing an impression and can be provided in a form that is suitable for electronic data processing.

The term “impression tray” is representative of a carrier element for the impression compound, and the present invention is consequently not restricted to the use of a carrier element in the form of a tray or to a configuration based on a dental impression tray in the conventional sense, but rather, as will be self-evident to a person skilled in the art, the form, shaping and dimensioning of the carrier element are governed by the intended use, to achieve which a person skilled in the art with knowledge of the present invention no longer has to exercise any independent inventive skill.

This makes it possible to capture dental structures, arrangements or shapes in the mouth or on or in the human body in data form digitally and three-dimensionally in a way that is easy, dependable and accurate.

The sensor devices are preferably designed to capture in a spatially resolved manner on the impression compound a
change in the radiation transparency and/or radiation absorption, in particular transparency to light and/or absorption of light,
change in the electrical conductivity,
change in the pressure, in particular by changes of the conductivity as a result of the change in pressure,
deformation,
change in cross section or change in thickness,
change in the electrical resistance and/or
change in the density and/or change in the distribution of foreign atoms, in each case in particular by changes of the electrical or optical conductivity as a result of the change in density and/or change in the distribution of foreign atoms.

It is further preferred if interface devices are coupled to the sensor devices on the output side, in order to pass on data generated by the latter in a form suitable for electronic data processing, the interface devices preferably comprising USB interface devices.

There are preferably also memory devices, in particular memory devices that can be decoupled, arranged downstream of the interface devices, preferably as a chip card or as a memory stick.

There are preferably also connecting devices, in particular wireless connecting devices, arranged downstream of the interface devices, preferably as Bluetooth®, infrared and/or radio devices.

The impression compound is, in particular, a homogeneous compound, which like a conventional impression compound is filled into the corresponding configuration of the dental impression tray, or generally the impression tray, before the latter is arranged in a likewise conventional manner in the patient's mouth and pressed onto the teeth. For example, the impression compound may also be sterilized after each use and then used again. The impression compound may also preferably have the following properties:
transparency to light
electrical conductivity
change in the conductivity due to pressure
measurement by deformation
change in cross section
increase in resistance
density and distribution of foreign atoms that determine the conductivity.

In accordance with the changes when it is pressed onto teeth or some other structure in the human body and the property thereof, the compound passes on data to the sensor devices on the surface and on the inner faces of the impression tray, in particular dental impression tray. The data thus obtained may, in particular, either be stored directly in the impression tray, in particular dental impression tray, or be transmitted by cable, USB or radio to a central PC, where they can then be used for further processing operations.

A further advantage is that the data can be sent online directly to a dental laboratory for further processing.

Another preferred development is that the impression tray is set up or acts as a carrier for x-ray film holders. In this case, the material of the impression tray may, furthermore, be designed such that it does not allow x-rays to pass through, at least in the regions or parts that are used in the area of measurement or treatment.

It is further preferred if time measuring devices are integrated in the impression tray, wherein in particular optical and/or acoustic signaling and/or indicating devices are assigned to the time measuring devices.

Yet another preferred configuration is that there is an integrated storage battery, which in particular can be charged, possibly via the USB port.

It is further of advantage that an ergonomically shaped handle is provided.

A further preferred embodiment is that there is a capturing unit and a recording unit with a base plate, a frame, an inner delimitation and the sensor devices. In this case, furthermore, the capturing unit and the recording unit may preferably be releasably connected to a handle (10), in particular by way of a U-shaped holding plate, and/or the surface of the impression tray or at least of the base plate, frame, inner delimitation and sensor devices, may comprise a coating or be finished in such a way that no bacteria adhere to it or bacteria on it are automatically destroyed, and/or at least the base plate, frame, inner delimitation and/or sensor devices are adjustable in size.

Furthermore, it may preferably be provided that there are heating devices, in particular in order to influence the flow behavior of the impression compound (14) or provide a sterilizing function of its own.

Yet a further preferable embodiment is that on the underside of the impression tray there is a registration for the opposing jaw to bite onto, and/or that the impression tray is designed such that it can be used to take impressions of the upper jaw and lower jaw simultaneously.

With the impression tray, it may also be preferably provided that, by grinding, the impression compound used, such as in particular clear plastic, can at the same time be used as an optical lens.

The sensor devices may also advantageously be designed to respond to an impression compound that contains one or more substances which only react in a specific way to light waves or react to specific light waves. In this case it is preferred if the sensor devices, the capturing unit and/or the recording unit is/are designed to provide the light waves and/or if the sensor devices are designed to allow the determination of a changed transmission or reflection behavior in the impression compound to be established when objects are pressed into it.

It may also be preferably provided that the impression compound is a transparent polyether, preferably with great hydrophilicity, or an impression compound based on polyether, A-silicone, C-silicone hydrocolloid, polysulfide and/or alginate.

Yet another preferred embodiment is that the impression compound is reusable.

It may preferably also be provided that, after taking an impression, the impression compound reacts to applied agents, such as for example sprays or liquids, in order to bring about a data transfer and/or storage.

A further preferred embodiment is that the impression compound is chosen such that its consistency is changed by supplied electrical energy.

Furthermore, it may preferably be provided that the impression compound is of such a nature that it is a memory compound and accordingly has a memory effect, in that after activation it reverts to its original shape.

In a further preferable embodiment there may also be a screen or display and/or input devices, such as for example keys.

A film may also preferably be provided, with which film the impression compound can be covered before an impression is taken, in order to prevent contact with saliva or mouth tissue/skin and/or to transmit data by deforming.

It is further preferred if a camera is provided in or on the impression tray, in order to record an image of the patient or at least reference points and add it/them to the jaw/tooth data determined.

The invention also provides a method for capturing structures, arrangements or shapes, such as preferably for capturing dental structures, arrangements or shapes in the mouth or in the human body, wherein a deformable impression compound is introduced into the mouth or body and a change of at least one physical property and/or variable of the impression compound is transmitted there in a spatially resolved manner directly to sensor devices when preparing an impression and is captured by the sensor devices and, furthermore, provided in a form that is suitable for electronic data processing.

This makes it possible to capture dental structures, arrangements or shapes in the mouth or in the human body in data form digitally and three-dimensionally in a way that is easy, dependable and accurate.

The sensor devices preferably capture on the impression compound a
change in the radiation transparency and/or radiation absorption, in particular transparency to light and/or absorption of light,
change in the electrical conductivity,
change in the pressure, in particular by changes of the conductivity as a result of the change in pressure,
deformation,
change in cross section or change in thickness,
change in the electrical resistance and/or
change in the density and/or change in the distribution of foreign atoms, in each case in particular by changes of the electrical or optical conductivity as a result of the change in density and/or change in the distribution of foreign atoms.

It is further preferred if interface devices are coupled to the sensor devices on the output side, in order to pass on data generated by the latter in a form suitable for electronic data processing, the interface devices preferably comprising USB interface devices.

Preferably, memory devices, in particular memory devices that can be decoupled, are also arranged downstream of the interface devices, preferably as a chip card or as a memory stick.

Furthermore, connecting devices, in particular wireless connecting devices, are preferably arranged downstream of the interface devices, preferably as Bluetooth®, infrared and/or radio devices.

Furthermore, in accordance with the changes when it is pressed onto teeth or some other structure in the human body and the property thereof, the compound may pass on data to the sensor devices on the surface and on the inner faces of the impression tray, in particular dental impression tray.

The data thus obtained may, with preference, either be stored directly in the impression tray, in particular dental impression tray, or be transmitted by cable, USB or radio to a central PC, where they can then be used for further processing operations, and/or the data may be sent online directly to a dental laboratory for further processing.

A further preferable method variant is that firstly a first impression is prepared with a first impression material, and then a second impression is prepared with, for example, additionally or alternatively a low-viscosity impression material, which when used in combination possibly in turn passes on information to the first impression material. In this case it may be further preferred to use the two impression materials with different impression trays.

However, it may preferably also be provided that the impression compound is composed of three different impression materials that cannot be mixed with one another, of different colors or different transmission and/or reflection properties, or that the impression compound consists of a number of films placed one on top of the other, in particular of different colors.

Yet a further preferred embodiment is that, to produce prostheses for parts of the teeth as a whole, dental structures, individual teeth and parts of teeth, firstly an impression of the existing state before a treatment is prepared, and the corresponding data are therewith determined, after that the treatment is performed, such as for example grinding down of a morbid tooth, an impression of the new state is once again prepared and the corresponding data of the new state are determined, and then, by means of matching and/or difference methods, a prosthesis, such as for example a crown or bridge, with the exact inner and outer shape and dimensions is produced from the data from the two impressions taken.

The invention also relates to a device for capturing a three-dimensional structure of the human or animal body, in particular a tooth or set of teeth, which comprises the following:
a carrier for an impression compound,
an impression compound arranged on the carrier,
at least one lighting unit, which is designed for radiating light into the impression compound, and
at least one sensor unit, which is designed for detecting light emerging from the impression compound and generating spatially resolved raw data therefrom.

The light emerging from the impression compound and detected by the sensor unit may be light which originates from an interaction between the impression compound and the irradiated light, light which is reflected by the structure to be measured or light which originates from a combination of these phenomena.

In one embodiment of the invention, the impression compound comprises at least one material which is selected from the group consisting of the fluorescent materials, the phosphorescent materials, the light-diffusing materials and the light-reflecting materials.

In one embodiment of the aforementioned measure, the impression compound comprises at least one material which is selected from the group consisting of the fluorescent materials and the phosphorescent materials, wherein the at least one lighting unit is designed for emitting light of a wavelength which lies in the range of excitation of the fluorescent materials and/or the phosphorescent materials.

In one embodiment of the invention, the impression compound is optically transparent in at least one wavelength range.

In one embodiment of the aforementioned measure, the lighting unit emits light of a wavelength which lies in a wavelength range of the optical transparency of the impression compound.

In one embodiment of the invention, the at least one lighting unit comprises a light source which is selected from the group consisting of LEDs, RGB-LEDs, OLEDs and laser LEDs.

In one embodiment of the invention, the at least one lighting unit is designed for projecting a pattern into the impression compound.

In one embodiment of the invention, the impression compound comprises a pattern which has been applied to it and/or incorporated in it.

In one embodiment of the invention, the at least one lighting unit is designed for emitting pulsed light.

In one embodiment of the aforementioned measure, the raw data contain spatially resolved light transit time data.

In one embodiment of the invention, the raw data contain spatially resolved brightness data.

In one embodiment of the invention, the at least one sensor unit comprises a multiplicity of glass fibers and at least one optical sensor, wherein one end of the glass fibers is respectively aligned with the impression compound and wherein a second end of the glass fibers is respectively aligned with the at least one optical sensor.

In one embodiment of the aforementioned measure, the at least one optical sensor is selected from the group consisting of CCD chips and CMOS chips.

In one embodiment of the invention, the device further comprises a memory unit for storing the raw data generated by the at least one sensor unit.

In one embodiment of the invention, the device further comprises a computing unit for generating image data from the raw data generated by the at least one sensor unit.

In one embodiment of the invention, the device further comprises an interface for passing on the raw data generated by the at least one sensor unit or the image data generated by the computing unit to a data processing unit.

Still further preferred and/or advantageous embodiments of the invention are provided by the claims and the combinations thereof as well as the present application documents as a whole, and in particular the explanations and representations of exemplary embodiments in the description and the drawing. Device and method features are also obtained from analogous implementation of features respectively specified with respect to methods and devices.

### BRIEF DESCRIPTION OF THE DRAWINGS

The invention is explained in more detail below merely by way of example on the basis of exemplary embodiments and with reference to the drawing, as follows.

FIG. 1 shows a schematic perspective representation of an upper jaw to explain the invention.

FIG. 2 shows a schematic perspective representation of an impression tray in the form of a dental impression tray with a capturing unit, recording unit, USB stick, radio unit and storage medium.

FIG. 3 shows a schematic perspective representation of the impression tray in the form of a dental impression tray from FIG. 2 filled with an impression compound.

FIG. 4 shows a schematic front-view representation of the upper jaw and the impression tray in the form of a dental impression tray from FIG. 3 and directly before taking an impression.

FIG. 5 shows a schematic plan-view representation of the negative impression of the upper jaw in the impression compound that is in the impression tray in the form of a dental impression tray from FIG. 3 after taking an impression.

FIG. 6 shows a schematic cross-sectional representation of the impression compound after taking an impression.

FIG. 7 shows a further schematic sectional representation of the impression compound that is in the impression tray in the form of a dental impression tray from FIG. 3 after taking an impression.

FIG. 8 shows a schematic plan-view representation of the negative impression of the upper jaw in the impression compound that is in the impression tray in the form of a dental impression tray from FIG. 3 after taking an impression, coinciding with FIG. 5.

FIG. 9 shows a schematic representation of the data in a PC.

FIG. 10 shows a schematic representation of a model produced in a production machine, such as a milling cutter, in particular a computer numerical control (CNC) milling cutter, by means of computer aided manufacturing (CAM) under the control of the PC.

FIG. 11 shows a schematic perspective representation of a finished model, which has been produced on the basis of the determined, obtained and processed data, in particular largely automatically.

FIG. 12 shows a schematic perspective representation of a further exemplary embodiment of the impression tray in the form of a dental impression tray.

FIG. 13 shows a schematic perspective representation of the further exemplary embodiment of the impression tray in the form of a dental impression tray from FIG. 12 with an additional detail.

FIG. 14 shows a schematic perspective representation of yet a further exemplary embodiment of the impression tray in the form of a dental impression tray.

FIG. 15 shows a schematic perspective representation of a detail of another exemplary embodiment of the impression tray in the form of a dental impression tray.

FIG. 16 shows a schematic lateral perspective representation of yet a further exemplary embodiment of the impression tray in the form of a dental impression tray.

FIG. 17 shows a schematic view from below of the impression tray in the form of a dental impression tray from FIG. 16.

FIG. 18 shows a schematic perspective representation of a further detail of another exemplary embodiment of the impression tray in the form of a dental impression tray.

FIG. 19 shows a schematic perspective representation of yet another exemplary embodiment of the impression tray in the form of a dental impression tray.

FIG. 20 shows a schematic perspective representation of yet another exemplary embodiment of the impression tray in the form of a dental impression tray.

FIG. 21 shows a schematic perspective representation of a further exemplary embodiment of the impression tray in the form of a dental impression tray in use on a patient.

FIG. 22 shows a schematic perspective representation of the exemplary embodiment according to FIG. 21 of the impression tray in the form of a dental impression tray.

FIG. 23 shows a carrier for an impression compound with an impression compound arranged on it.

FIGS. 24a-c show an impression compound provided with a pattern, respectively before, during and after taking an impression.

FIG. 25 shows a schematic, perspective representation of a further exemplary embodiment of the impression tray in the form of a dental impression tray.

FIG. 26 shows a schematic, perspective representation of the impression tray from FIG. 25.

FIGS. 27a-b show a schematic representation of a geometrical measuring method that can be used in the impression tray from FIG. 25.

FIG. 28 shows a further schematic representation of a geometrical measuring method that can be used in the impression tray from FIG. 25.

### DESCRIPTION OF PREFERRED EMBODIMENTS

On the basis of the exemplary embodiments and examples of use that are described below and represented in the drawing, the invention is explained in more detail merely by way of example, i.e., it is not restricted to these exemplary embodiments and examples of use or to the respective combinations of features within individual exemplary embodiments and examples of use. Method and device features are also respectively obtained by analogy from descriptions of the device and method.

Individual features that are specified and/or represented in connection with actual exemplary embodiments are not restricted to these exemplary embodiments or the way in which they are combined with the other features of these exemplary embodiments, but may, to the extent that is technically feasible, be combined with any other variants, even if they are not treated separately in the present documents, and in particular with features and configurations of other exemplary embodiments.

The same reference numerals in the individual figures and illustrations of the drawings designate components that are the same or similar or act in the same or a similar way. Representations in the drawing also clearly disclose features that are not provided with reference numerals, irrespective of whether or not such features are subsequently described. On the other hand, features which are contained in the present description but are not visible or represented in the drawing can also be readily understood by a person skilled in the art.

Device and method features are also respectively obtained from graphic and written representations of methods and devices.

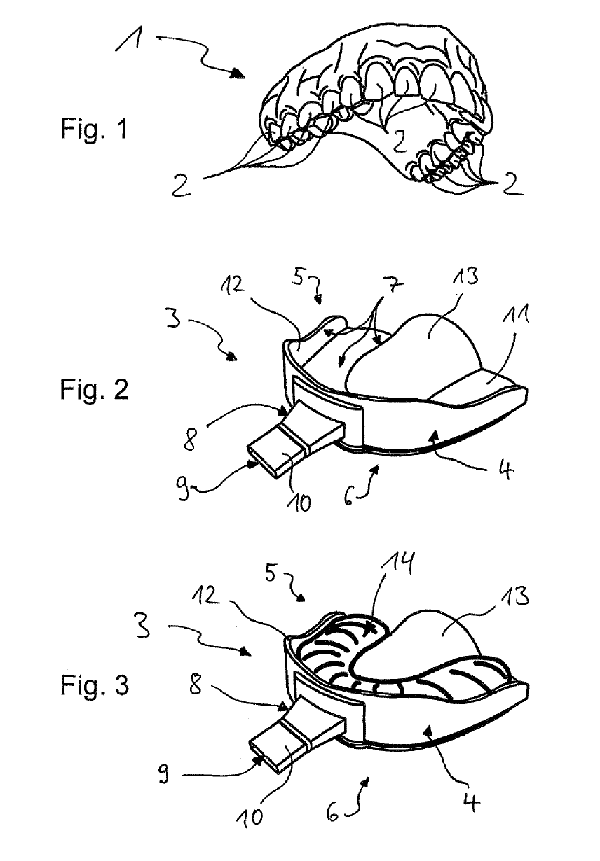

FIG. 1 shows a schematic perspective representation of a human upper jaw 1 with teeth 2, given by way of example of many applications of the invention, in a single representation for better clarity, to explain the invention.

Shown in a schematic perspective representation in FIG. 2 is an impression tray 3 in the form of a dental impression tray 4, which acts as it were as a capturing unit 5 and recording unit 6, for which purpose the impression tray 3 comprises sensor devices 7, and an extension 8 with a USB port 9, in order to be able, by way of the latter, to directly transmit data obtained to for example a PC (not represented). In addition or as an alternative to the USB port 9, the data transmission possibility may also be provided by an integrated or connectable radio unit (not represented) or, for example, by integrated or connectable Bluetooth® or infrared devices (not represented). The radio unit or Bluetooth® devices may possibly be alternatively accommodated, or likewise accommodated, in the extension 8. A further possibility for data transmission may be achieved by using an exchangeable storage medium (not represented), which is formed as the extension 8, accommodated therein or can be connected thereto or to suitable ports (not represented) formed in some other way of the impression tray 3, such as for example a conventional USB stick (not represented) or a chip card (not represented). Furthermore, the extension 8 acts at the same time as a handle 10, by which the impression tray 3 can be gripped for introduction into a human oral cavity and alignment and placement therein on a jaw to be captured as a whole or in part, such as for example the upper jaw 1 from FIG. 1, which makes it easier to handle the impression tray 3 and minimizes the number of parts that are used. Moreover, the extension 8 may be removable from the impression tray 3.

The impression tray 3 in the form of the dental impression tray 4 comprises a base plate 11, a frame 12 and an inner delimitation 13, and the sensor devices 7 are assigned to the base plate 11, the frame 12 and/or the inner delimitation 13 according to the type of design and mode of operation of the actual configuration, which will be discussed in more detail below. To this extent, the dental impression tray 4 exactly resembles a conventional dental impression tray, which is of advantage since this allows it to be handled in an accustomed way. The base plate 11, the frame 12 and the inner delimitation 13 are consequently shaped for introduction and use in the oral cavity (not visible) and to match the upper jaw 1.

FIG. 3 is a schematic perspective representation of the impression tray 3 in the form of the dental impression tray 4 from FIG. 2 filled with a suitable impression compound 14 in a conventional manner, i.e., such as with a conventional elastic impression compound for the existing impression-taking technique.

The special feature of the impression tray 3, or in the present example of the dental impression tray 4, is thus the combination of deformable impression compound 14 and sensor devices 7. This combination is chosen such that a change of at least one physical property and/or variable of the impression compound 14 is captured in a spatially resolved manner by means of the sensor devices 7 when preparing an impression, for example of the upper jaw 1 or of at least one tooth 2 from FIG. 1, and is provided in a form that is suitable for electronic data processing, so as to obtain data from which 3D data can be determined, for example of the upper jaw 1 or of at least one tooth from FIG. 1, which 3D data then serve for the computer-aided production of prostheses.

The sensor devices 7 preferably capture on the impression compound 14 a
change in the radiation transparency and/or radiation absorption, in particular transparency to light and/or absorption of light,
change in the electrical conductivity,
change in the pressure, in particular by changes of the conductivity as a result of the change in pressure,
deformation,
change in cross section or change in thickness,
change in the electrical resistance and/or
change in the density and/or change in the distribution of foreign atoms, in each case in particular by changes of the electrical or optical conductivity as a result of the change in density and/or change in the distribution of foreign atoms.

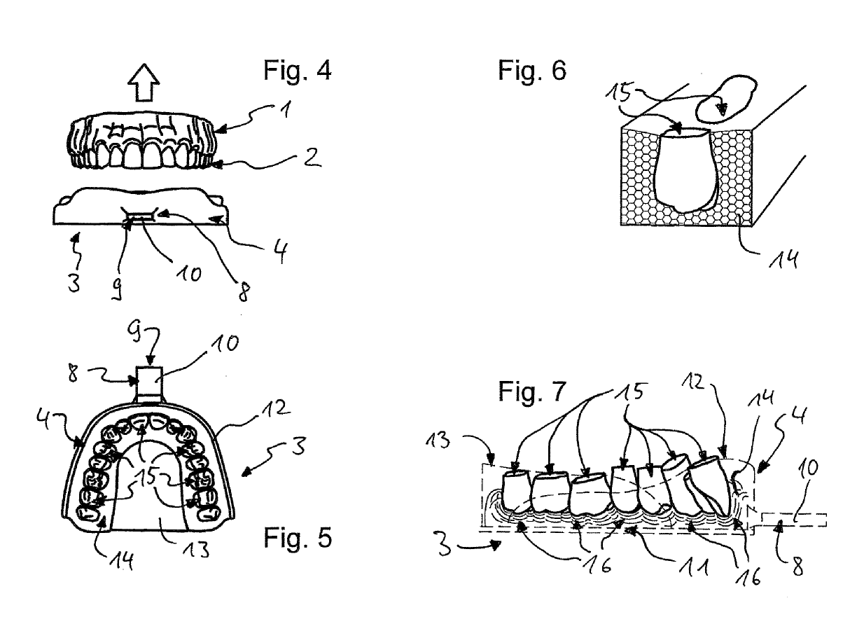

FIG. 4 shows a schematic front-view representation of the upper jaw 1 and the impression tray 3 in the form of the dental impression tray 4 from FIG. 3, directly before taking an impression, which is achieved, in the same way as in the preparation of previously customary impressions, in the case of the upper jaw 1 by the impression tray 3 being pressed onto the upper jaw 1 from below, so that the teeth 2 of the upper jaw 1 press into the impression compound 14. Also in the case of conventional impression compounds, it is ensured that there is sufficient fluidity, at least in an initial time of preparing the impression, so that the impression compound also penetrates into intermediate spaces (cannot be seen) during the pressing in of the teeth 2, which also preferably applies to the impression compound 14 of the present invention. In the case of conventional impression compounds, however, curing must in any event take place before the conventional impression compound together with the impression tray 3 can be released from the upper jaw 1 and removed, in order that the mold taken from the teeth 2 of the upper jaw 1 in the conventional impression compound is preserved, so that the plaster model of the teeth 2 of the upper jaw 1 that is customary in the prior art can then be produced with this mold.

In FIG. 5, the negative impression of the upper jaw 1 in the impression compound 14 that is in the impression tray 3 in the form of the dental impression tray 4 from FIG. 3 is illustrated in a schematic plan-view representation after taking an impression, hollow spaces 15 having been created in the impression compound 14 in accordance with the teeth 2 of the upper jaw 1 from which the impression has been taken. FIG. 6 shows the impression compound 14 in a schematic cross-sectional representation on its own after taking an impression, i.e., with impressed hollow spaces 15 in accordance with the teeth 2 of the upper jaw 1 from which the impression has been taken, and FIG. 7 shows an impression of part of the upper jaw 1 from FIG. 1 in a further schematic sectional representation of the impression compound 14 that is in the impression tray 3 in the form of the dental impression tray 4 from FIG. 3 after taking an impression. The deformations within the impression compound 14 are illustrated by the curved lines 16 in FIG. 7, which deformations are produced by the deforming of the impression compound 14 as a result of the pressing in of the teeth 2 of the upper jaw 1 when the impression is taken and lead to the changing of physical properties and/or variables of the, or in the, impression compound 14, which in turn are captured by the sensor devices 7, whereby the functions of the sensor devices 7 in conjunction with the impression compound 14 as a capturing unit 5 and recording unit 6 are achieved. Instead of being symbolic of deformations, the curved lines 16 may also be understood as symbolic of individual regions of pressure, regions of transparency, regions of conductivity, regions of concentration, etc., depending on the type of combination of impression compound 14 and sensor devices 7, and depending on the effect that is used in the impression compound 14 by the pressing in of the teeth 2 of the upper jaw 1 and is captured by the sensor devices 7.

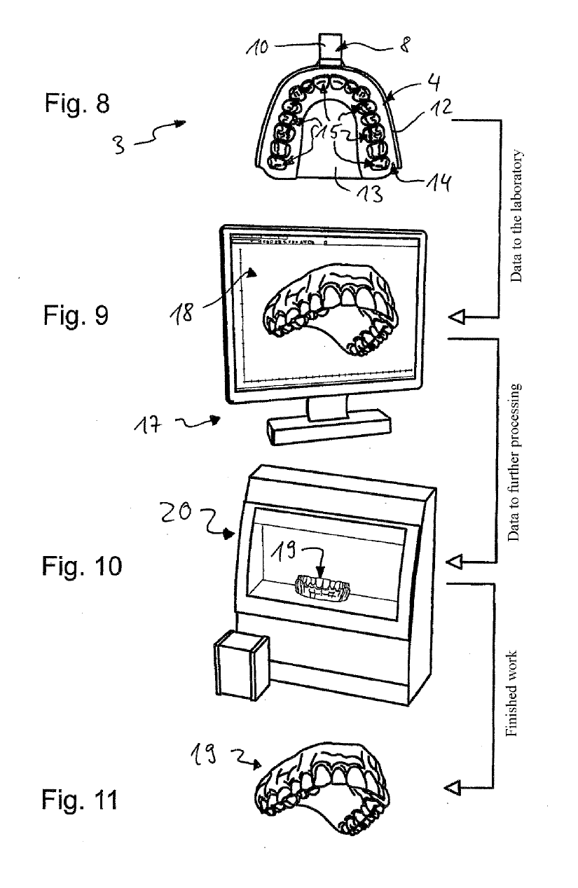

As in FIG. 5, in FIG. 8 there is shown a schematic plan-view representation of the negative impression of the upper jaw 1 in the impression compound 14 that is in the impression tray 3 in the form of the dental impression tray 4 from FIG. 3 after taking an impression. It is then possible for the data obtained by the sensor devices 7 in conjunction with the impression compound 14 to be displayed, edited and processed on a PC 17 by suitable software, as illustrated by the schematic representation of the 3D data of the upper jaw 1 from FIG. 1, obtained by taking an impression, in an edited graphic representation on the screen 18 of the PC 17 in FIG. 9. The editing and processing of the data obtained and determined by the sensor devices 7 in conjunction with the impression compound 14, that is to say the data from the capturing unit 5 and the recording unit 6, yield a result which is illustrated by a schematic representation of a created model 19 in a 3D milling cutter 20 or similar device (not shown). The production of the model preferably takes place for example by means of a CNC milling cutter by CAM under the control of the PC 17. This then allows the finished model 19 of the upper jaw 1 from FIG. 1 to be produced in accordance with the schematic perspective representation in FIG. 11 largely automatically, and in particular completely automatically, consequently having been created on the basis of the determined, obtained and processed data without manual laboratory work, as would be required after taking an impression in the conventional way.

The invention makes it possible that the 3D data required for the automatic production of the model 19 of the upper jaw 1 are obtained directly in the patient's mouth—or generally at the actual site for which a prosthesis is to be produced. In this respect it does not matter whether the data are already provided by the capturing unit 5 and the recording unit 6 of the sensor devices 7 in conjunction with the impression compound 14 as general 3D data, or, a data format obtained therefrom is only converted into actual 3D data that can be put to further use for example, after transmission to the PC 17 by USB link, radio, Bluetooth®, infrared or the like, for which the PC 17 readily provides the required computing capacity, or indeed has to be chosen appropriately. It is also not decisive whether a PC 17 performs, or can perform, further processing of the data from the impression tray 3 on site, i.e., at the dental or gnatho-orthopedic surgery, or whether this PC 17 only serves for recording data from the impression tray 3 and passing data on to a separate computer, such as for example a PC 17 in a laboratory, which may easily take place online, so that the data required for producing the model can in any event get to a laboratory, where the production of the model 19 is performed, quickly, inexpensively and dependably, which is illustrated by the indication “Data to the laboratory” from FIG. 8 to FIG. 9.

However, the further processing of the data from the impression tray 3 through to the completion of the model 19 may unreservedly also take place decentrally in the dental or gnatho-orthopedic surgery, so that a patient can possibly even be provided straightaway with the model 19 or, to be more correct, with the prosthesis produced. In any event, the step from computer-aided data acquisition to automated production is illustrated by the indication “Data to further processing” between FIGS. 9 and 10. The indication “Finished work” from FIG. 10 to FIG. 11 illustrates that the finished model 19, which is symbolic of a prosthesis to be produced, which is at the end of the process of capturing through to production.

It will be readily appreciated that the procedure described above with the impression tray 3 according to the invention not only makes the production of entire jaw models or prostheses much easier and quicker in comparison with the entire prior art, but that it is possible in particular to produce prostheses of individual teeth and parts of teeth or groupings of individual teeth and parts of teeth, such as for example bridges and crowns, and these will be the most frequent applications for example in odontology, which can be seen as constituting a huge potential for use.

In terms of the method, to produce prostheses for parts of the teeth as a whole, dental structures, individual teeth and parts of teeth, firstly an impression of the existing state before a treatment is prepared, i.e., the corresponding data are determined. After that, the treatment, such as for example grinding down of a morbid tooth, is performed. This is followed by once again preparing an impression of the new state, i.e., the corresponding data of the new state are determined. By means of matching and difference methods, which are all customary and known, a prosthesis, such as for example a crown or bridge, with the exact inner and outer shape and dimensions can be produced from the data from the two impressions taken.

As a further development of the invention, the impression tray 3 may, for example, also be used as a carrier for x-ray film holders. The material of the impression tray 3 may then be designed such that it does not allow X-rays to pass through, at least in the regions or parts that lie in the area of measurement or treatment.

For easily monitoring time-relevant or time-critical events when taking an impression, in the impression tray 3 there may be integrated time-measuring devices (not shown), to which optical and/or acoustic signaling and/or display devices (not shown) are assigned. For these and other electrically operated devices in or on the impression tray 3, it is of advantage if it has the USB port 9, since, by way of the latter, it is also easily possible for example to charge a storage battery that is preferably used to operate the electrical devices.

The impression-taking process may take place in a number of stages. For example, first a first impression may be prepared with a first impression material of the “coarse form”, and then a second impression with, for example, additionally or alternatively low-viscosity impression material, which when used in combination possibly in turn passes on information to the first impression material, providing a fine data record. Such a procedure may be of advantage to reduce the amounts of data per impression taken, which makes it possible for the 3D data ultimately obtained to be produced more easily and quickly, and under some circumstances also more accurately. The two impression materials may also be used with different impression trays 3. To this extent, the term “impression compound 14” is representative of one or more materials that may be used at the same time or one after the other. The impression compound 14 may, for example, also be composed of three different impression materials that cannot be mixed with one another, of different colors, different transmission and/or reflection properties, so that specific data and possibly information can be obtained from each material. The impression compound 14 may in this case also consist of a number of films placed one on top of the other, for example of different colors.

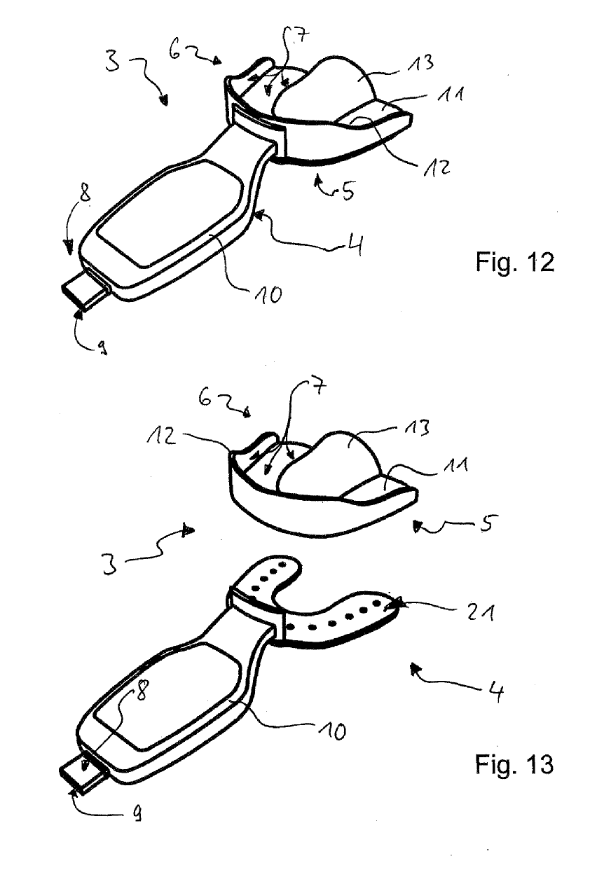

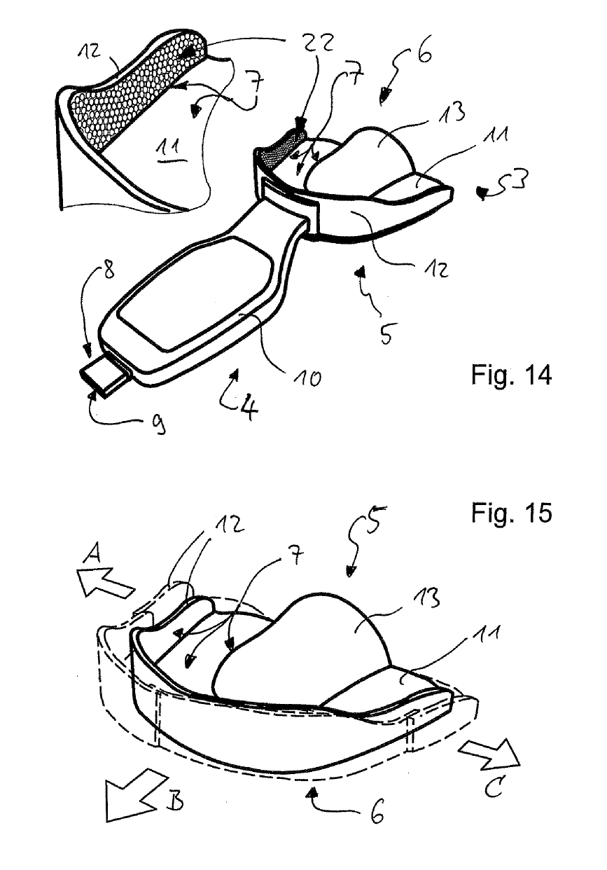

In FIGS. 12 and 13, a further design of the impression tray is illustrated, it having been considered important here to have an ergonomically shaped handle 10. As is clear from the comparison of FIGS. 12 and 13, it is also provided in the case of this impression tray 3 that the capturing unit 5 and the recording unit 6 with the base plate 11, frame 12, inner delimitation 13 and sensor devices 7 are releasably connected to the handle 10 by way of a preferably U-shaped holding plate 21. Consequently, all the technical elements that come into contact with the oral cavity of a patient can be removed from the rest of the impression tray 3 and can also be separately cleaned and sterilized. The surface of the impression tray 3, or at least of the base plate 11, frame 12, inner delimitation 13 and sensor devices 7, may be coated or finished in such a way that no bacteria adhere to it or bacteria on it are automatically destroyed, as illustrated by the coating 22 in FIG. 14.

According to another exemplary embodiment, illustrated in FIG. 15, the impression tray 3 may be designed such that at least the base plate 11, frame 12, inner delimitation 13 and/or sensor devices 7 are adjustable in size, in order to achieve optimizing adaptation to circumstances pertaining to individual patients, as symbolized by the arrows A, B and C.

The impression tray 3 may also be heatable, in order to influence the flow behavior of the impression compound 14, or to provide a sterilizing function of its own.

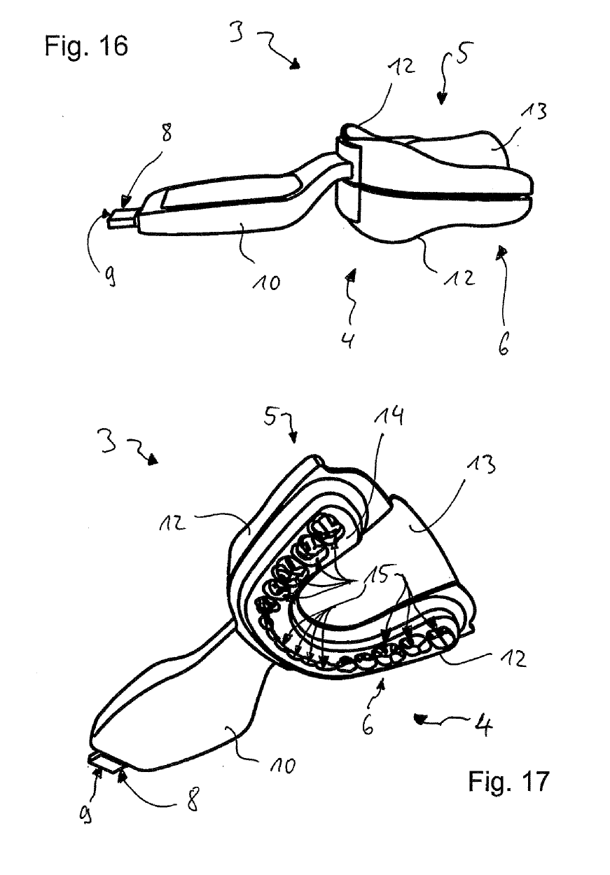

On the underside of the impression tray 3 there may be a registration for the opposing jaw to bite onto, in order that the jaws can be assigned to one another during the later processing of the data obtained in the PC. However, the impression tray 3 may also be designed such that, as revealed by FIGS. 16 and 17, it can be used to take impressions of the upper jaw and lower jaw simultaneously.

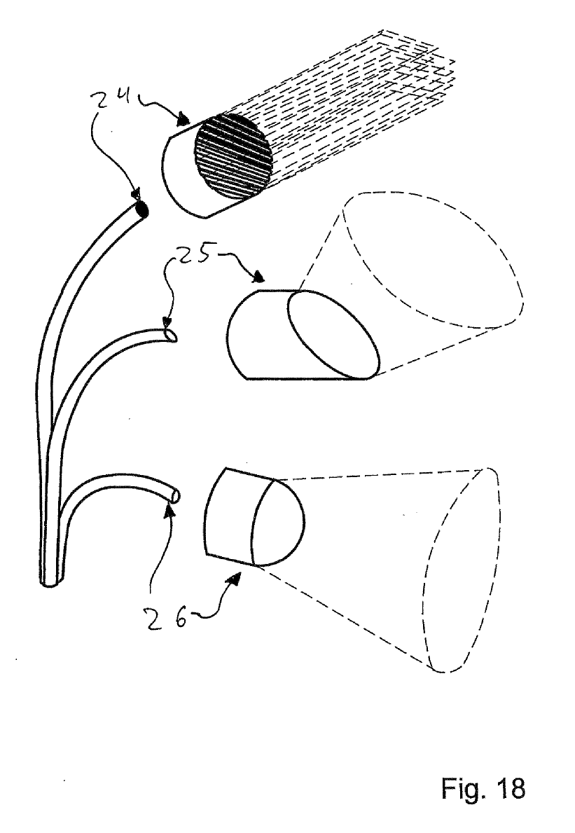

The impression tray 3 may also be designed such that, by grinding, the impression compound 14 used, such as for example clear plastic, can at the same time be used as an optical lens. Such lenses 23, 24 and 25, as represented by way of example in a number of variants in FIG. 18, may be designed such that, like the lens 23 for example, they project or record a striped pattern onto the item to be identified. It is also possible in this case to use glass fibers, the free ends of which are ground so as to obtain lenses 24, which, by having a beveled light-exiting area, capture different regions when turned, or lenses 25, which have a beam-widening effect.

The impression compound 14 may contain one or more substances which only react in a specific way to light waves or react to specific light waves that are provided by the sensor devices 7, or generally the capturing unit 5 and/or the recording unit 6, in order to allow changed transmission or reflection behavior in the impression compound 14 to be established in a spatially resolved manner, as indeed provided by the invention, as a result of the teeth 2 of the upper jaw 1 being pressed into it. If a different impression compound 14 were used, one not containing such an adjuvant, the sensor devices 7 could not determine data, or no data could be determined with their aid.

The impression compound 14 may be transparent polyether, preferably with great hydrophilicity. The impression compound 14 may also be based on polyether, A-silicone, C-silicone hydrocolloid, polysulfide and/or alginate. The impression compound 14 is preferably transparent in accordance with the effect used that is to be captured by the sensor devices 7, so that light refraction, degree of transparency or transparency to specific wavelengths can be used.

Although the impression compound 14 may be such that it can be cleaned and sterilized for further use, reusability is not absolutely necessary.

An impression compound 14 which, after taking an impression, reacts to applied agents, such as for example sprays or liquids, in order to bring about a data transfer and/or storage, may also be used. The impression compound 14 may also be chosen such that its consistency is changed by supplied electrical energy. The impression compound 14 may also be of such a nature that it is a memory compound and accordingly has a memory effect, and that after activation it reverts to its original shape.

There are consequently numerous available effects which, individually or in combination, enable the sensor devices 7 to sense changes of the impression compound 14. At the same time, allowance can also be made for further properties of the impression compound 14, in order to make it possible for it to be used, and impressions to be taken, as easily, quickly and accurately as possible.

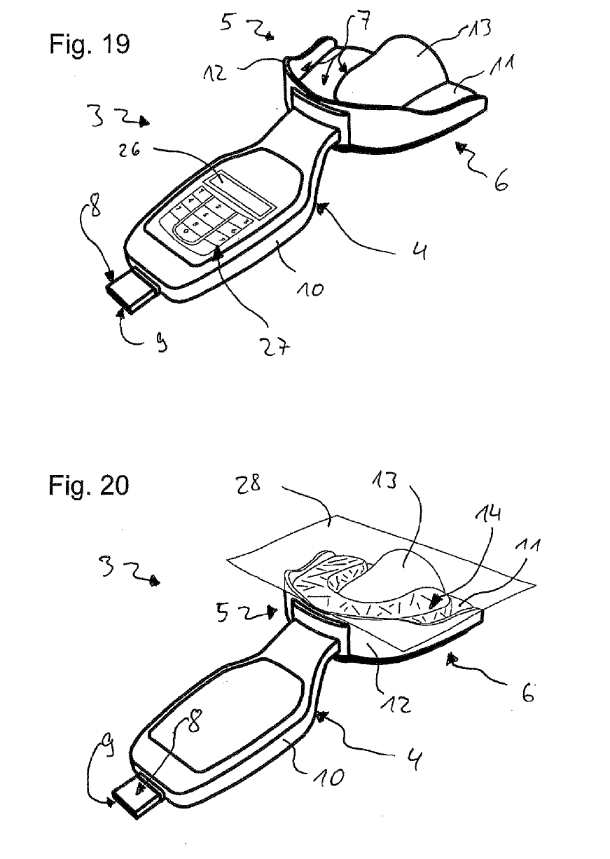

The impression tray 3 may itself also be provided with a screen 26 or display and with input devices, such as in particular keys 27, in order to check and facilitate applications, as made clear by the representation of FIG. 19.

Furthermore, according to the exemplary embodiment of FIG. 20, a film 28 may be provided, with which film the impression compound 14 can be covered before an impression is taken, in order to prevent contact with saliva or mouth tissue/skin and/or to transmit data by deforming.

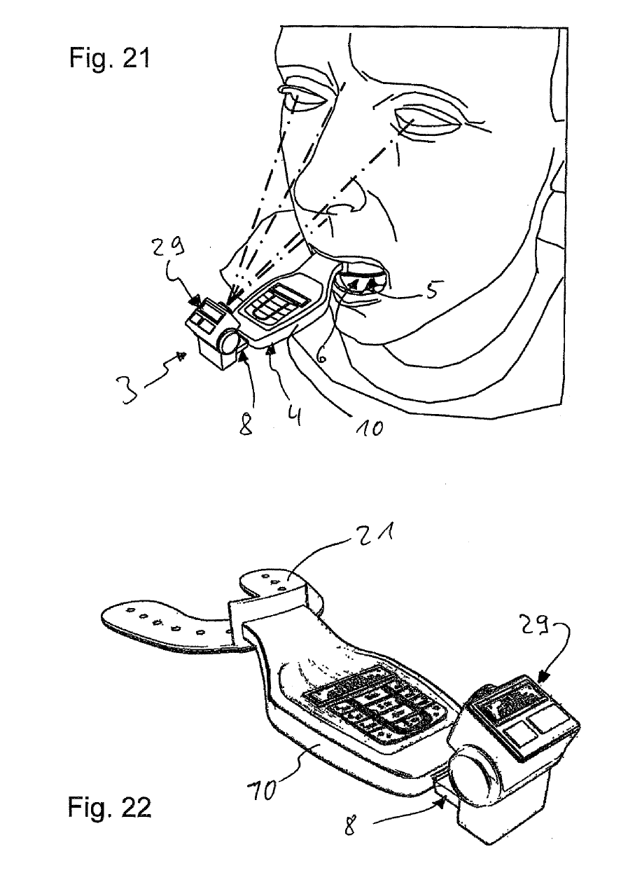

Furthermore, a camera 29 may be provided in or on the impression tray 3, in order to record an image of the patient or at least reference points and add it/them to the jaw/tooth data determined, whereby an assignment of the data obtained in relation to the head of the patient as a whole is additionally made possible, as illustrated by FIGS. 21 and 22.

Although the above description has made reference predominantly to use of the impression tray 3 according to the invention in dental treatment applications, the technology according to the invention is not restricted to such applications, but can, with knowledge of the present invention, also be advantageously used for procuring data by taking impressions on animals, plants, open body parts, the inner ear, other cavities as well as machine parts and components without requiring any independent inventive skill.

In the description and in the drawing, the invention is presented on the basis of the exemplary embodiments merely by way of example and is not restricted to them, but rather comprises all variations, modifications, substitutions and combinations that a person skilled in the art can take from the present documents, in particular within the scope of the claims and the general representations in the introductory part of this description as well as the description of the exemplary embodiments and their representations in the drawing, and can combine with his knowledge of the art and the prior art, in particular the disclosure contents of the prior publications specified at the beginning. In particular, all the individual features and configurational possibilities of the invention and the various ways in which they can be embodied can be combined.

A further subject of the invention is a non-curing impression compound.

In dental practice it is common to use impression materials for the anatomical modeling of teeth and jaw portions for the evaluation, diagnosis, planning, and monitoring of the accuracy of fit of preservative, prosthetic, and gnatho-orthopedic work. Captured in this case is the approximate form of the jaw and of the teeth in an anatomical snapshot, by the taking of what is called an anatomical impression. After the anatomical impression has been poured up with plaster suspension, the dentist then obtains what is called the study model, diagnostic model, documentation model, working and planning model. For the representation of the opposing jaw in the case of more extensive prosthetic work so called, opposing-jaw models are also produced, which are obtained by taking impressions using alginates.

One class of dental impression materials is represented by the addition-crosslinking silicones, which are presently in use as precision impression materials for producing ultraprecise working models for the fabrication of replacement teeth. The properties of such compounds compositions are described, for example, in the standards ISO 4823 and ADA 19. Addition-crosslinking silicones are described, for example, in U.S. Pat. No. 4,035,453.

Commercial addition-crosslinking silicone impression compounds are typically present in a two-part form, consisting of a base paste and a catalyst paste, in which the reactive components are spatially separate from one another for reasons of stability. The materials cure after the two pastes have been mixed up in precisely defined volume proportions.

Also used are condensation-crosslinking silicones, so-called C-silicones.

A disadvantage of the reactive impression compounds is that in general they have to be mixed up from two components prior to use, with the need to observe precise metering of the amounts. The requisite mixing in the dental surgery and the cure time constitute a hindrance to the work of the dentist.

Other widespread dental impression compounds are based on reactive polyethers. Impression compounds of this kind are described, for example, in DE 19753456 A1 and EP 0865784 A2. Also in use are impression compounds with natural polymers such as alginates or agar, which cure by gelling.

All of the dental impression compounds employed to date are curing compounds, which are inconvenient to manage. Furthermore, the shelf life of the reactive compositions is limited.

The object of the invention is to provide an alternative impression material and impression-taking method.

The object has been achieved by impression compounds which comprise substances without reactive groups or which, in view of the absence of added catalysts, do not cure under the conditions of use.

Impression compounds of this kind are based on a liquid phase of greater or lesser viscosity, comprising no substances having reactive groups, or comprising substances which, on account of the composition of the impression compound, do not crosslink under the conditions of use. The impression compound contains no catalyst for a crosslinking or curing reaction. The impression compound is based in general on substances which are liquid at room temperature, such as liquid silicones (organopolysiloxanes), polyethers, hydrocarbons (for example oils), vegetable oil or liquid mixtures. Referred to below as the liquid phase. Dissolved in the liquid phase there may be corresponding or other solid substances.

Liquid silicones are, for example, silicone oils such as linear, nonreactive polydimethylsiloxanes.

Polyethers are, for example, polyethylene glycols, polypropylene glycols or mixed polyethers (for example composed of tetrahydrofuran structural units and ethylene oxide and/or propylene oxide structural units). In the liquid phase there may be, for example, liquid polyethers combined with solid polyethers.

Suitable hydrocarbons, straight-chain or branched, preferably saturated, nonvolatile liquid hydrocarbons, are, for example, liquid paraffin, n-paraffins, isoparaffins. Petrolatum, an ointment-like mixture of solid and liquid hydrocarbons, may be used as a liquid phase.

The liquid phase advantageously comprises inert diluents. As inert diluent, use is made of polyether polyols, such as, for example, polypropylene glycols or mixed polyetherols with tetrahydrofuran units and/or with ethylene oxide and/or propylene oxide units, polyester polyols, such as, for example, polycaprolactone diols and polycaprolactone triols, polycarbonate diols, aliphatic ester, oils, fats, waxes, aliphatic hydrocarbons, araliphatic hydrocarbons, and mono- or polyfunctional esters of mono- or polybasic acids, such as, for example, phthalic acid or citric acid, or esters or amides of alkylsulfonic acids and arylsulfonic acids.

The impression compound generally comprises the liquid phase, one or more fillers, optionally further adjuvants, auxiliaries and dyes or pigments.

The base substance of the liquid phase, and the fillers, are preferably transparent to light, advantageously in a wide wavelength range, for example in the range from 200 to 700 nm or 300 to 700 nm. Transparency or partial transparency to certain types of radiation, examples being radiation in the UV range, radiation in the range of visible light (vis range) or in the UV/vis range, radiation in the infrared range (IR range), in the near infrared range, or even x-radiation, is of interest for particular applications, especially in combination with optical methods for the 3D capture of impressions.

Particular impression compounds or their basic composition consist(s) of an inert liquid such as silicone oil or liquid paraffin and colorless metal oxides, in particular precipitated or fumed silica. They are suitable for application with optical sensors.

A silicone-based impression compound according to the invention comprises, for example, the following constituents:

(a) one or more organopolysiloxanes, preferably without reactive groups,

(b) filler,

(c) optionally further adjuvants, auxiliaries and dyes,

(d) optionally hydrophilizing agents,

the impression compound containing no catalyst for a crosslinking reaction.

The amount of component (a) is generally 30% to 80% by weight, preferably 60% to 80% by weight, based on the total mass of the impression material. The components of the impression material and their amounts are selected such that the compound commonly has a Shore A hardness, determined in accordance with DIN 53505, of less than 45, preferably <40, and a consistency, determined in accordance with ISO 4823, of 31 to 39 mm.

Preferred as component (a) are diorganopolysiloxanes having terminal triorganosiloxy groups.

The polymer preferably has a viscosity at 25° C. of between 200 and 200000 mPa·s, more preferably 1000 to 10000 mPa·s.

Particularly preferred are linear polydimethylsiloxanes or mixtures thereof with the indicated viscosity ranges.

Suitable components (a) are polymeric organosiloxanes without reactive substituents. These are preferably linear, branched or cyclic organopolysiloxanes in which all of the silicon atoms are surrounded by oxygen atoms or by monovalent hydrocarbon radicals, it being possible for the hydrocarbon radicals to be substituted or unsubstituted.

The hydrocarbon radicals are, for example, methyl, ethyl, C2-C10 aliphatics, trifluoropropyl groups, and aromatic C6-C12 substituents.

Particularly preferred as component (a) is a mixture of silicones having a relatively high viscosity (for example 1000 to 10000 mPa·s at 25° C.) and of silicones having a relatively low viscosity (for example 50 to 1000 mPa·s at 25° C.). Low-viscosity silicones are, for example, polydimethylsiloxanes which have trimethylsiloxy end groups. The amount of low-viscosity silicone is, for example, 1% to 40% by weight, preferably 5% to 40% by weight, more preferably 15% to 30% by weight, based on the total mass of component (a).

In order to generate a hydrophilic impression compound it is advantageous to add an agent which imparts hydrophilic nature, or hydrophilizing agent, component (d), thereby inducing better wettability of the overall composition in the moist oral environment and hence a better flow-on behavior of the pastes. The hydrophilizing agents do not have reactive groups. Suitable hydrophilizing agents are preferably nonincorporable wetting agents from the group of the hydrophilic silicone oils, which are described in WO 87/03001 and in EP-B-0 231 420, the relevant disclosure content of which is hereby to be incorporated by reference. Preference is given, furthermore, to the ethoxylated fatty alcohols described in EP-B-0 480 238. Preferred hydrophilizing agents, moreover, are the polyethercarbosilanes known from WO 96/08230. Preference is also given to the nonionic, perfluoroalkylated, surface-active substances that are described in WO 87/03001. Likewise preferred are the nonionic surface-active substances described in EP-B-0 268 347, i.e., the nonylphenol ethoxylates, polyethylene glycol monoesters and diesters, sorbitan esters, and also polyethylene glycol monoethers and diethers that are recited therein. The amounts used of the hydrophilizing agents are 0.1% to 10% by weight, based on the total weight of all of the components, preferably 0.2% to 2% by weight and more preferably 0.3% to 1% by weight.

The fillers which can be used as component (b) include nonreinforcing fillers having a BET surface area of up to 50 m²/g, such as quartz, cristobalite, calcium silicate, zirconium silicate, montmorillonites such as bentonites, zeolites, including the molecular sieves, such as sodium aluminium silicate, metal oxide powders, such as aluminium oxides or zinc oxides or the mixed oxides thereof, barium sulfate, calcium carbonate, gypsum, powdered glass and powdered plastics. Possible fillers also include reinforcing fillers having a BET surface area of more than 50 m²/g, such as, for example, fumed or precipitated silica, and mixed silicon aluminium oxides with a large BET surface area. The stated fillers may be hydrophobized, by means, for example, of treatment with organosilanes and/or organosiloxanes, or by the etherification of hydroxyl groups to alkoxy groups. It is possible to use one kind of filler; it is also possible to use a mixture of at least two fillers. The grain distribution is preferably selected such that there are no fillers present with grain sizes >50 μm. The total amount of the fillers (b) lies in the range from 10% to 80%, preferably 30% to 60%, with the amounts of filler being selected such that a Shore A hardness of the compound of <45 is not exceeded.

Particularly preferred is a combination of reinforcing and nonreinforcing fillers. In this case, the reinforcing fillers are in quantity ranges from 1% to 10% by weight, in particular 2% to 5% by weight. The balance in the stated overall ranges, i.e., 9% to 70% by weight, in particular 28% to 55% by weight, is formed by the nonreinforcing fillers.

Preference as reinforcing fillers is given to pyrogenically prepared, highly disperse silicas, which have been rendered hydrophobic preferably by surface treatment. The surface treatment may take place, for example, with dimethyldichlorosilane, hexamethyldisilazane, tetramethylcyclotetrasiloxane or polymethylsiloxanes. The surface areas of suitable fumed silicas are preferably >50 m²/g, in particular 80 to 150 m²/g. The presence of the surface-treated fumed silicas contributes to the adjustment of the consistency and to the improvement of the sag resistance of the pastes. At amounts of <1% by weight, it is generally not possible to ascertain any noticeable effect on the sag resistance; amounts of >10% by weight lead in general to excessive thickening of the pastes, meaning that sufficient fluidity can no longer be obtained. Suitable products are described in, for example, the brochures from Degussa, now Evonik Degussa (Aerosil Products, Pigments Text Series, No. 11, 5th edition, 1991, on page 79, and also from Cabot Corp. (Cabosil products, “CAB-O-SIL® Fumed” silica in Adhesives and Sealants, Cabot, 1990).

Particularly preferred nonreinforcing fillers are quartzes, cristobalites and sodium aluminium silicates, which may be surface-treated. The surface treatment may take place in principle with the same methods as described in the case of the reinforcing fillers.

A further filler is diatomaceous earth or kieselguhr. It consists of the very manifoldly formed silica skeletons of single-cell, microscopically small algae (diatoms) which live in fresh or salt water. The materials are extracted usually by surface mining and are also referred to as infusorial earth, mountain flour or bacilli earth. Types of diatomaceous earth used with preference are employed in calcined form. Preferred types of diatomaceous earth are, for example, the products with the trade names “Celatom” (sold for example by Chemag), “Cellite 219”, “Cellite 499”, “Cellite 263 LD”, “Cellite 281” and “Cellite 281 SS” from Johns-Manville, and also “Diatomite 104”, “Diatomite CA-3”, “Diatomite IG-33”, “Diatomite 143”, “Diatomite SA-3”, “Diatomite 183” from Dicallite, and also the “Clarcel” products from Ceca.

Furthermore, the impression compounds according to the invention advantageously comprise, as component c), dyes, preferably fluorescent dyes, pigments or finely divided metals, and also antioxidants, preservatives, release agents. The compounds of the invention comprise such adjuvants in amounts of preferably 0% to 20% by weight, more preferably from 0.1% to 1% by weight.

The impression compound advantageously comprises microbicidal or disinfectant agents such as Chloramine T, Chlorhexidine, copper or silver in fine distribution. Self-disinfecting materials are described in DE 19814133 A1, hereby incorporated by reference. Disinfectants are present in the impression compound at, for example, 3 to 7 percent by weight.

The impression compound advantageously comprises colorants, which comprise dyes, fluorescent dyes, phosphorescent materials, pigment, luminescence systems, in particular chemiluminescence systems, and substances or polymers with chromophoric groups. Dyes in the narrower sense are soluble in the liquid phase; insoluble dyes are referred to as pigments.

Colorants present in the impression compound are used advantageously in combination with an optical sensor system. When the colorant is distributed throughout the impression compound, the colorants are employed in amounts such that the impression compound is still translucent. The colorant-containing impression compound ought still to have good transmission for the measuring radiation used in an optical measurement system at, for example, a path length of 1 cm. Where the colorants are used in a coating of the impression compound, very high colorant densities are also used. Colorants are present in the impression compound at, for example, 1 to 5 percent by weight.

Dyes are, for example, indigo, indigotin, betanoin, chlorophyll a, chlorophyll b, chlorophyll c1, chlorophyll c2, chlorophyll d, green S, patent blue V (Na salt), patent blue V (Ca salt), brilliant blue FCF, brilliant black BN, brown HT, riboflavin, zeaxanthin, tartrazine, quinoline yellow S, yellow orange S, carotene, curcumin, lutein, annatto, canthaxanthin, capsanthin, lycopene, lithol rubine, azo rubine, amaranth, allura red. Dyes are present in the impression compound at, for example, 1 to 5 percent by weight.

Fluorescent dyes are, for example, fluoresceins, rhodamines, coumarins, berberin, chini, DAPI, Nile red, allophycocyanin, indocyanin green, stilbene, porphyrins (haems, chlorophylls, etc.), especially luminol, perylene, coelenterazine, latia luciferin, lucioptery, photinus luciferin, fluorescein, eosin Y. Fluorescent dyes are present in the impression compound at, for example, 5 to 20 percent by weight.

Phosphorescent materials are mostly crystals with a low level of admixture of an extraneous substance which disrupts the lattice structure of the crystal. It is usual to use sulfides of metals of the second group, and also zinc, and to admix small amounts of heavy metal salts (for example zinc sulfide with traces of heavy metal salts).

Pigments used are, for example, organic dyes insoluble in the liquid phase, metal salts, effect pigments, finely divided metals (for example Cu, Ag, Au).

Pigments are, for example, titanium dioxide, iron oxide (yellow), iron oxide (red), iron oxide (black).

Pigments are present in the impression compound at, for example, 1 to 5 percent by weight.

Colorants may also be polymers with chromophoric groups. Such polymers may be, for example, modified silicones or polyethers.

Chemiluminescence (also chemoluminescence) is a process in which, through a chemical reaction, electromagnetic radiation in the visible light range that is not of thermal origin is emitted. The best-known chemiluminescence systems are, for example, the oxidation of luminol by hydrogen peroxide in the presence of iron ions or manganese ions, peroxyoxalate chemiluminescence, and the chemiluminescence of 1,2-dioxetanes. The chemiluminescence systems are preferably in pressure-sensitive coatings of the impression compound or of an impression body, the reaction components being present in—for example—microencapsulated form. For example, very small reagent quantities of the components of a chemiluminescence system are enveloped with wax or other customary substances by customary methods of microencapsulation. The microcapsules may be fixed, for example, directly as a thin layer on the surface of an impression body or of a thin film (for example adhesive bonding, adhesion, electrostatically, etc.) or may be admixed to a coating material (for example impression compound). As a result of pressure, particularly in the course of taking an impression, the components of the chemiluminescence system are released, and the chemiluminescence reaction can take place in the region of the impression. The light that is emitted in the chemiluminescence reaction can be detected by a sensor system.

Pressure-sensitive layers, coatings or films (for example covering film) may be constructed generally with microencapsulated colorants or reagents for color formation (for example color change on alteration in pH value). Not all of the components of a system need be microencapsulated. It is possible, for example, for one component to be present free in a layer or in the impression compound. For the coating of an impression body it is advantageous to use a composition which comprises a film-forming polymer. Serving as film-forming polymers are, for example, polyvinyl alcohol, polyvinyl acetate, polyvinylpyrrolidone, polyamide, polyarylsulfone and copolymers thereof.

Film-forming polymers are used, for example, in dissolved form or as a dispersion. A “film-forming polymer” is a polymer that has the capacity, alone or in the presence of a film-formation auxiliary, to form a continuous and adhering film on a substrate, which may be a film or the surface of a layer of an impression compound or of an impression body. The film-forming polymer is, for example, a polyurethane polymer. Film-forming polymers and film-forming compositions are described in, for example, DE 60105246 T2, DE 69736168 T2 and EP 0447964 B1, hereby incorporated by reference. The composition may comprise, for example, one or more colorants, one or more radiation-absorbing polymers (polymers with chromophore), conductive particles, magnetic particles or microencapsulated substances, especially reagents.

Impression compounds with a different liquid phase are produced analogously.

The impression compound is preferably translucent. This is important for an optical capture of an impression. For such an application, the impression compound must have a sufficient transparency for light, preferably in the wavelength range from 300 to 700 nm.

The impression compound is preferably sterilizable and temperature-stable at up to 200° C.

Advantages of the impression compound:

chemical stability, simple management, cost-effective, reusable.

One or more impression compounds are used for producing an impression body.

Impression bodies are shaped structures comprising one or more impression compounds, which may have additional parts or modifications. The impression body generally has a carrier or is provided for accommodation in a carrier. The carrier is, for example, dish-shaped.

In the dental sector, impression compounds and impression bodies are used, for example, for impression trays. In an impression body, it is advantageous for impression compounds of different natures and properties to be combined. For example, soft and harder impression compounds are combined, in order to be firmer in the outer region and softer in the impression region (FIG. 23 with impression compounds 14′, harder, and 14, softer).

Impression compounds may be layered, examples being horizontal layers of light and dark (in alternation), of different colors.

Between layers in an impression body there may be films used with a grid pattern or with other patterns.

It is advantageous to use a cover film, in particular with a pattern such as grid lines, with impression bodies. This is shown in FIG. 24. FIG. 24a shows an impression body with an impression compound 14 and a cover film 28 with a grid line pattern, before taking an impression. In FIG. 24b, an intermediate phase during the taking of an impression is shown, and in FIG. 24c the finished impression. The change in the grid line pattern in the region of the impression may serve as an aid in the three-dimensional capture of the impression.

Examples of suitable cover films include elastic films of polyethylene-LD and PVC, of the kind employed in freshness retention films, and films of polyurethane.

The surface of an impression body may also be printed directly with a pattern.

The capturing system is explained below.

The capturing system is preferably a capturing system which determines the three-dimensional shape of an impression with the aid of sound, in particular ultrasound, or radiation, in particular light. This may take place on the basis of various operating principles or measuring principles: radar measurement with sound or radiation, geometrical measurement and absorption of radiation. Advantageously, two or all of the measuring principles mentioned may be combined. Radar measurement uses, for example, the reflection of a radiation pulse, in particular a light pulse, on the surface of the impression body or an object. (Distance measurement by way of the transit time of a reflected beam or reflected sound). The geometrical measurement uses the distance dependence of the size of an incoming radiation cone (for example incoming light cone) of a bundle of optical fibers. In absorption measurement, the path length dependence of the absorption of a reflected light beam in a medium is evaluated. The capturing system comprises more than one measuring point, preferably three or more measuring points and, particularly preferably, a multiplicity of spatially distributed measuring points. Such a capturing system comprises at least one energy source (for example a radiation source or a sound source, in particular an ultrasound source), at least one sensor or receiver for the energy (for example an image sensor or an array of sensors or receivers) and a control and evaluation unit. An optical capturing system is particularly preferred. In the case of an optical capturing system, optical fibers with one or more image sensors are advantageously used. The optical fibers are generally connected to the image sensor, wherein, particularly advantageously, each pixel or a group of pixels of the image sensor is assigned an optical fiber, which ends in the direct proximity of said pixel or group of pixels. The capturing system preferably comprises one, two or more carriers for an impression compound or is connected to one or more such carriers. The other end of the optical fibers, remote from the image sensor, is preferably arranged in the region of the carrier. There may also be one or more image sensors arranged directly in the region of a carrier. The measuring points are, for example, the ends of the optical fibers or pixels of an image sensor in the region of the carrier.

The carrier is, for example, a kind of trough or dish, in particular U-shaped in the case of dental applications. Such carriers are, for example, those known as impression trays.

Glass fibers or polymer fibers (POF) serve as optical fibers. The optical fibers are generally used as bundles. Sorted optical fibers are preferably used. The glass fiber also comprises optical fibers with a fiber core of quartz. Polymer fibers are, for example, PMA/PMMA fibers. Particularly flexible are polyurethane fibers.

Used for example as image sensors are CCD or CMOS sensors, as are used in digital cameras or cameraphones. For example, a CMOS sensor chip (dimensions: 12.5×12.5 mm) with a resolution of 6 million pixels with a pixel size of 5 μm is used. Generally, a bundle of optical fibers is coupled directly to the sensor. Preferably, a pixel is assigned an optical fiber, with all or only some of the sensor pixels being used. The bundle of fibers is advantageously connected to the sensor chip by a plug-in system. The image sensors are generally used without color filters in front of the pixels, that is to say the image sensors are generally operated monochromatically.

For the detection and capturing of x-radiation, a fluorescent film or similar aid (with fluorescent or phosphorescent substances) may be arranged between the carrier and the impression compound or impression body, in front of an image sensor on the carrier or optical fibers.

Preferably a number of radiation sources are used, advantageously also different radiation sources. In the case of the optical capturing systems, light-emitting diodes (LEDs) are used for example as the radiation source. LEDs which emit light in the UV range, visible range or IR range, that is to say in the range of, for example, 200 nm to 900 nm, are used. LEDs of different ranges or wavelengths are advantageously combined. The LEDs are preferably operated in a pulsed or clocked manner. Particularly advantageously, light pulses of different wavelengths are used for the measurement, the light pulses of the different wavelengths being emitted simultaneously or successively. Laser light (for example a laser diode), which is in particular deflected by means of controllable micromirrors, may also be used as radiation, whereby the micromirrors cannot be arranged in the impression compound because of being able to move. The radiation pulses of different radiation sources (for example LEDs) may be emitted from different locations simultaneously or successively. This generally takes place in accordance with a special program, and the radiation sources are correspondingly controlled by a control unit.

The radiation pulses may be emitted in a directed or undirected manner. In the case of directed radiation emission, the beam may be deflected by means of controllable mirrors, in particular micromirrors. What is known as DLP® technology (DLP®: Digital Micromirror Device™) from Texas Instruments is particularly suitable for this. The radiation source, for example LEDs, may be arranged directly on the carrier for the impression compound or the impression body, for the example impression tray. The radiation may, however, also be directed to the impression compound or impression body, for example, via optical fibers or mirrors. In a preferred embodiment, the same or different LEDs are distributed over the carrier, for example as a row or kind of strip on the bottom of an impression tray. The radiation sources are preferably arranged directly under the impression compound or the impression body, which are thus illuminated upwardly from below.

With the aid of one or more radiation sources, a pattern, for example grid lines or grid points, can be advantageously projected onto the surface (including the surface pressed in by the impression) of the impression compound or the impression body. Specially configured LEDs may be used for the projection of the patterns.

The preferred optical sensor system uses the reflection of a radiation, in particular of radiation pulses or pulsed radiation, for precisely capturing the contours of the impression of a body in an impression compound or an impression body with the aim of producing a faithful and true-to-scale three-dimensional model of the body.

The capture may take place during the formation of the impression, while the impression is being taken and/or after the impression has been taken, the impression-forming body being present or removed. For capturing the contour of an object or body, all the phases of forming an impression can be used, that is to say sequences from the beginning of taking the impression to the finished impression may be recorded. For example, up to 500 images per second may be recorded with the image sensor.

From the measured values, a pseudo-plastic contrast image is created by means of an evaluation unit. At the same time, a three-dimensional relief is thereby determined. This system makes it possible to produce, in particular, a tooth replacement required for dental treatment completely in a computer-aided manner.

An optical sensor system is generally used with a completely or partially transparent impression compound. Instead of an impression compound, a liquid (for example water, oil, silicone oil or polyether) may also be used if no impression of a body is required and the body is three-dimensionally captured directly. For dental applications, the approach of taking an impression is more advantageous, since the gums are pushed back somewhat by the impression compound.

For an absorption measurement, the impression compound advantageously contains a colorant, particularly advantageously a dye or a fluorescent dye, which are dissolved in the liquid phase. Colorants or the radiation is/are chosen such that the radiation can be absorbed by the colorant. In the case of a dye, the beam is attenuated along the length of the path by the medium as a result of absorption. A beam reflected at the boundary surface of the impression compound passes along a path of a shorter length in the region of the impression after the impression has been taken than before the impression is taken. Therefore, the path length is locally changed by the impression, and consequently the absorption is changed. This effect may be used in addition or as an alternative to a radar measurement for determining distances or thicknesses. By analogy, the change in the fluorescence may be captured as a measure of the change in the local path length. Using different beams (for example light of different wavelengths), locally different radiation sources, different dyes or colorants and variation of the pulse lengths, it is possible to generate a wealth of data that can be used for 3D capture.

Measurements with the system during or after an impression is taken are compared in the evaluation with measurements before the impression was taken. A calibration of the system with the aid of an object of which the position and dimensions are precisely known is advantageous. Fixed points or auxiliary structures on the surface of the impression body (for example a film with grid lines as a covering before and during the taking of an impression) may also be used in the calibration as well as the capture.

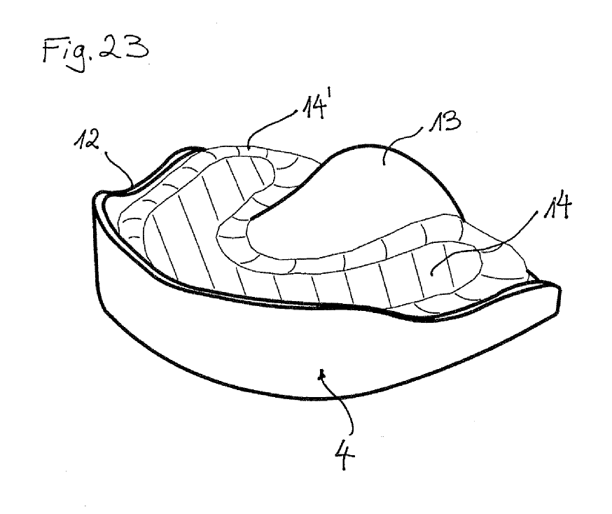

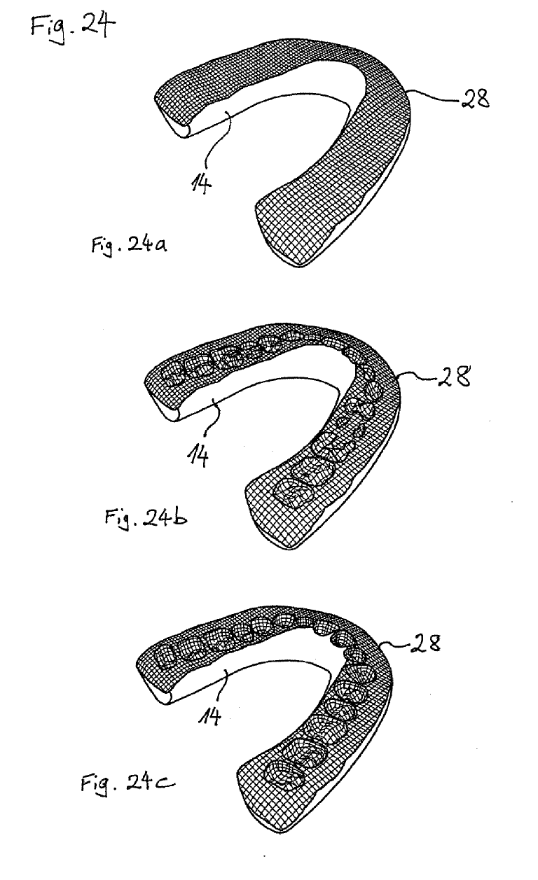

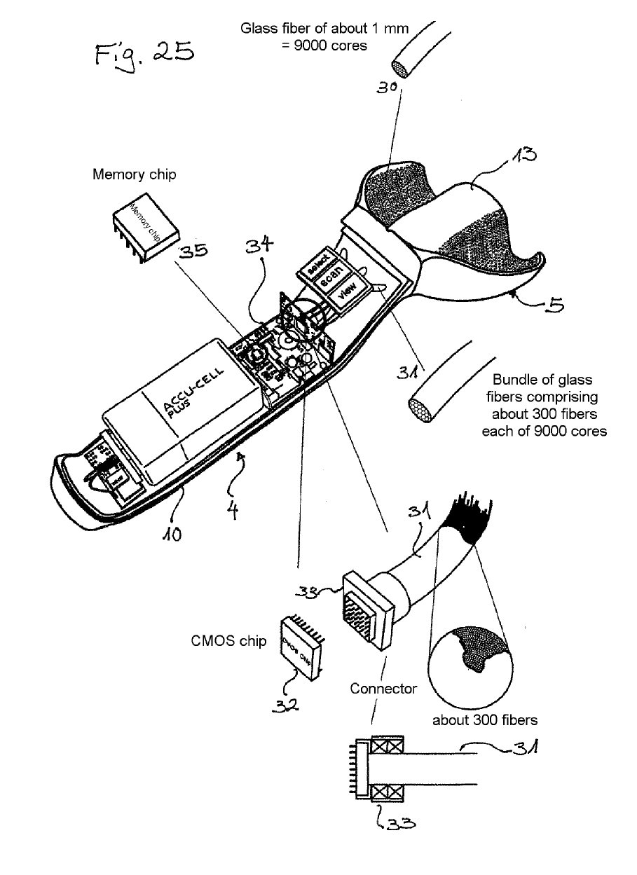

A further example of a system for capturing a three-dimensional structure of the human or animal body, in particular a tooth or set of teeth, as well as how its functions are explained below on the basis of FIGS. 25 to 28. The system comprises the following:
a carrier for an impression compound,
an impression compound arranged on the carrier,
at least one lighting unit, which is designed for radiating light into the impression compound, and
at least one sensor unit, which is designed for detecting light emerging from the impression compound and generating spatially resolved raw data therefrom.

For reasons of overall clarity, the impression compound is not represented in FIG. 25.

In such a system, the impression compound may comprise at least one material which is selected from the group consisting of the fluorescent materials, the phosphorescent materials, the light-diffusing materials and the light-reflecting materials.

In such a system, the impression compound may comprise at least one material which is selected from the group consisting of the fluorescent materials and the phosphorescent materials, wherein the at least one lighting unit is designed for emitting light of a wavelength which lies in the range of excitation of the fluorescent materials and/or the phosphorescent materials.

In such a system, the impression compound may be optically transparent in at least one wavelength range.

In such a system, the lighting unit may emit light of a wavelength which lies in a wavelength range of the optical transparency of the impression compound.

In such a system, the at least one lighting unit may comprise a light source which is selected from the group consisting of LEDs, RGB-LEDs, OLEDs and laser LEDs.

In such a system, the at least one lighting unit may be designed for projecting a pattern into the impression compound.

In such a system, the impression compound may comprise a pattern which has been applied to it and/or incorporated in it.

In such a system, the at least one lighting unit may be designed for emitting pulsed light.

In such a system, the raw data may contain spatially resolved light transit time data.

In such a system, the raw data may contain spatially resolved brightness data.

In such a system, the at least one sensor unit may comprise a multiplicity of glass fibers and at least one optical sensor, wherein one end of the glass fibers is respectively aligned with the impression compound and wherein a second end of the glass fibers is respectively aligned with the at least one optical sensor.

In such a system, the at least one optical sensor may be selected from the group consisting of CCD chips and CMOS chips.

In such a system, the device may further comprise a memory unit for storing the raw data generated by the at least one sensor unit.

In such a system, the device may further comprise a computing unit for generating image data from the raw data generated by the at least one sensor unit.

In such a system, the device may further comprise an interface for passing on the raw data generated by the at least one sensor unit or the image data generated by the computing unit to a data processing unit.

FIG. 25 shows an example of a dental impression tray 4 with a capturing unit 5 and a handle 10. Optical waveguides 30, for example glass-fiber light guides with a large number of cores (individual fibers, for example 9000), are grouped together in bundles of glass fibers 31, in the example 300 glass fibers 30 being contained in one bundle of glass fibers 31. Each individual core represents a measuring point. The measuring points (ends of the glass fiber cores) are arranged and distributed on the inner face of the dish-shaped carrier with inner delimitation 13. The bundle of glass fibers 31 is connected to the image sensor 32, for example a CMOS sensor, by means of a plug-in connection with a connector 33. Each core of the glass fibers is assigned in a defined manner to a pixel of the image sensor. The light information (light intensity) of each core is captured by the image sensor 32 during a measurement. The measured values are stored with the aid of the control and memory device 34 with memory chip 35 and can later be transferred via a USB interface to a PC for evaluation. The carrier with the measuring points is referred to as the capturing unit 5. The capturing unit 5, the optical waveguide 30 or bundle of optical waveguides 31, the image sensor 33 with recording electronics and the control and memory device 34 represent the capturing system. The radiation sources or light sources for the emission of light pulses are formed by a row of LEDs, which are arranged in the region of the bottom of the depression in the carrier (base plate) and are not depicted in FIG. 25. Likewise not depicted is the impression compound or the impression body in the inner region of the depression of the carrier.

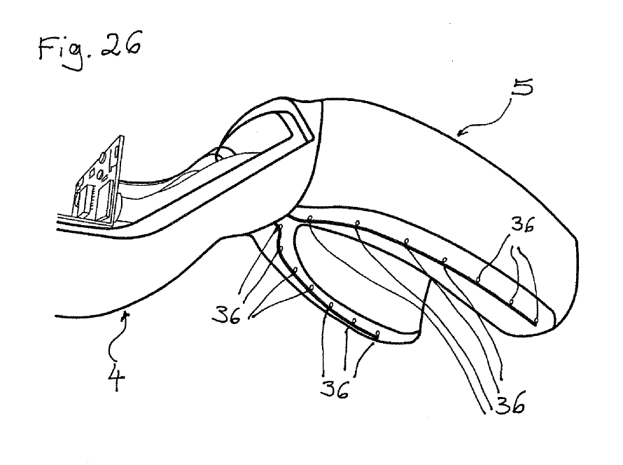

FIG. 26 shows the capturing unit 5 from FIG. 25 with the LEDs 36 as the radiation source for the optical capturing system. The LEDs 36, in FIG. 26 there are 14 of them, are arranged on the bottom of the carrier and emit light into the inner region of the carrier, into the impression compound or an impression body.

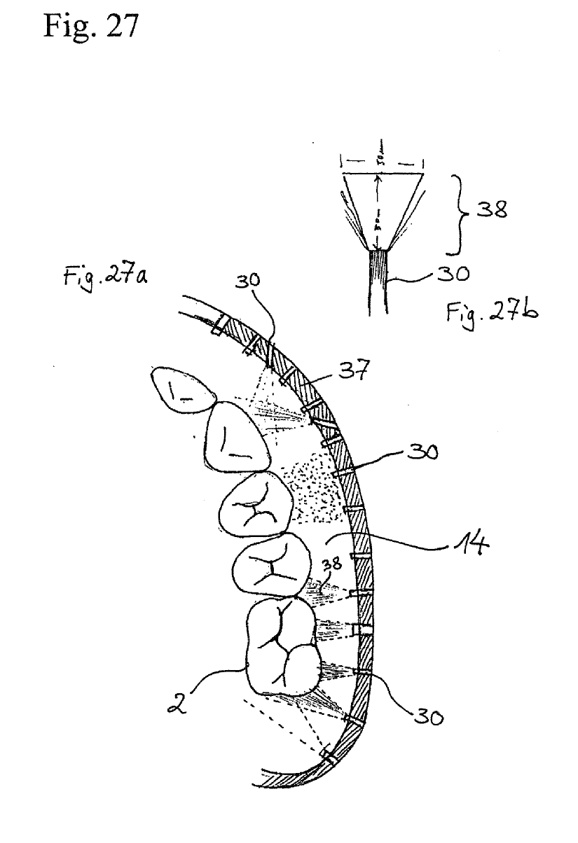

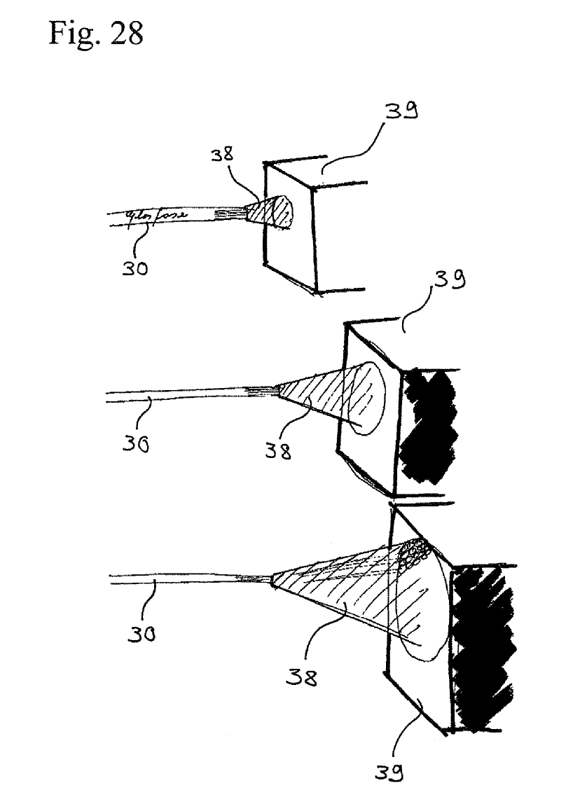

In FIGS. 27 and 28, the geometrical measuring principle is explained.

In FIG. 27a, the arrangement of teeth 2, glass fibers 30 and carrier wall 37 is represented in a schematic and greatly simplified form. Between the carrier wall 37 and the teeth 2 is the impression compound 14. Each glass fiber 30 has a light-entry cone 38, which depends on the shape and finish of the end of the glass fiber. For example, glass fibers 30 with a fixed light-entry angle of 45° are used (FIG. 27b). The ends of the glass fibers 30 are arranged in different alignments in the carrier wall 37, in order to capture the contour of the teeth or of the impression as well as possible from all sides.

In FIG. 28, it is illustrated how the light-entry cone 38 of the glass fibers 30 (for example with a diameter of about 1 mm and 600 cores or individual fibers) increases with the distance from an object 39 (for example a tooth 2). This means that the size of the light-entry cone 38 represents a measure of the distance of the object 39. As the light cone increases in size, the captured area of the object 39 increases, and consequently so does the amount of light collected by the glass fiber 30.

What is claimed is:

1. An impression tray, such as in particular a dental impression tray, which carries a deformable impression compound in order to prepare an impression of arrangements, shapes and/or dimensions, in particular in or on a human body, preferably in a mouth, and further preferred an impression of at least part of a tooth or of dental structures, further comprising sensor devices by means of which a change of at least one physical property and/or variable of said impression compound can be captured in a spatially resolved manner when preparing an impression and can be provided in a form that is suitable for electronic data processing.

2. The impression tray, such as in particular a dental impression tray, of claim 1, whereby said sensor devices are designed to capture in a spatially resolved manner on said impression compound at least one of
change in radiation transparency or radiation absorption, in particular transparency to light or absorption of light,
change in electrical conductivity,
change in pressure, in particular by changes of conductivity as a result of said change in pressure,
deformation,
change in cross section or change in thickness,
change in electrical resistance,
change in density or change in distribution of foreign atoms, in each case in particular by changes of electrical or optical conductivity as a result of said change in density or change in said distribution of foreign atoms.

3. The impression tray, such as in particular a dental impression tray, of claim 1, whereby interface devices are coupled to said sensor devices on an output side, in order to pass on data generated by said sensor device in a form suitable for electronic data processing, whereby said interface devices preferably comprise USB interface devices.

4. The impression tray, such as in particular a dental impression tray, of claim 3, further comprising memory devices, in particular memory devices that can be decoupled, arranged downstream of said interface devices, preferably selected from chip cards and memory sticks.

5. The impression tray, such as in particular a dental impression tray, of claim 3, further comprising connecting devices, in particular wireless connecting devices, arranged downstream of said interface devices, preferably selected from Bluetooth®, infrared and radio devices.

6. The impression tray, such as in particular a dental impression tray, of claim 1, whereby said impression compound is a homogeneous compound, which like a conventional impression compound is filled into a corresponding configuration of said dental impression tray before said dental impression tray is arranged in a likewise conventional manner in a patient's mouth and pressed onto teeth.

7. The impression tray, such as in particular a dental impression tray, of claim 6, whereby said impression compound can also be sterilized after each use and then used again.

8. The impression tray, such as in particular a dental impression tray, of claim 1, whereby the impression compound has at least one of the following properties:
transparency to light
electrical conductivity
change in conductivity due to pressure
measurement by deformation
change in cross section
increase in resistance
density and distribution of foreign atoms that determine conductivity.

9. The impression tray, such as in particular a dental impression tray, of claim 1, whereby said compound, in accordance with said changes when it is pressed onto teeth or some other structure in a human body and a property thereof, passes on data to said sensor devices on a surface and on inner faces of said impression tray.

10. The impression tray, such as in particular a dental impression tray, of claim 1, whereby said data obtained are either stored directly in said tray or are transmitted by cable, USB or radio to a central PC, where they can then be used for further processing operations.

11. The impression tray, such as in particular a dental impression tray, of claim 1, whereby said data are sent online directly to a dental laboratory for further processing.

12. The impression tray, such as in particular a dental impression tray, of claim 1, whereby said impression tray is set up or acts as a carrier for X-ray film holders.

13. The impression tray, such as in particular a dental impression tray, of claim 12, whereby a material of said impression tray is designed such that it does not allow X-rays to pass through, at least in the regions or parts that are used in an area of measurement or treatment.

14. The impression tray, such as in particular a dental impression tray, of claim 1, further comprising time measuring devices which are integrated in said impression tray.

15. The impression tray, such as in particular a dental impression tray, of claim 14, further comprising optical and/or acoustic signaling and/or indicating devices which are assigned to said time measuring devices.

16. The impression tray, such as in particular a dental impression tray, of claim 1, further comprising an integrated storage battery, which in particular can be charged, possibly via an USB port.

17. The impression tray, such as in particular a dental impression tray, of claim 1, further comprising an ergonomically shaped handle.

18. The impression tray, such as in particular a dental impression tray, of claim 1, comprising a capturing unit and a recording unit with a base plate, a frame, an inner delimitation and said sensor devices.

19. The impression tray, such as in particular a dental impression tray, of claim 18, whereby said capturing unit and said recording unit are releasably connected to a handle by way of a preferably U-shaped holding plate.

20. The impression tray, such as in particular a dental impression tray, of claim 18, whereby a surface of said impression tray or at least of said base plate, frame, inner delimitation and sensor devices, has a coating or is finished in such a way that no bacteria adhere to it or bacteria on it are automatically destroyed.

21. The impression tray, such as in particular a dental impression tray, of claim 18, whereby at least said base plate, frame, inner delimitation and/or sensor devices are adjustable in size.

22. The impression tray, such as in particular a dental impression tray, of claim 1, further comprising heating devices, in particular in order to influence the flow behavior of said impression compound or provide a sterilizing function of its own.

23. The impression tray, such as in particular a dental impression tray, of claim 1, whereby on an underside of said impression tray there is a registration for an opposing jaw to bite onto.

24. The impression tray, such as in particular a dental impression tray, of claim 1, whereby said impression tray is designed such that it can be used to take impressions of an upper jaw and a lower jaw simultaneously.

25. The impression tray, such as in particular a dental impression tray, of claim 1, whereby, by grinding, said impression compound used, such as in particular clear plastic, can at the same time be used as an optical lens.

26. The impression tray, such as in particular a dental impression tray, of claim 1, whereby said sensor devices are designed to respond to an impression compound that contains one or more substances which only react in a specific way to light waves or react to specific light waves.

27. The impression tray, such as in particular a dental impression tray, of claim 26, whereby said sensor devices, said capturing unit and/or said recording unit is/are designed to provide said light waves.

28. The impression tray, such as in particular a dental impression tray, of claim 26, whereby said sensor devices are designed to allow changed transmission or reflection behavior in said impression compound to be established when objects are pressed into it.

29. The impression tray, such as in particular a dental impression tray, of claim 1, whereby said impression compound is a transparent polyether, preferably with great hydrophilicity, or an impression compound based on polyether, A-silicone, C-silicone hydrocolloid, polysulfide and/or alginate.

30. The impression tray, such as in particular a dental impression tray, of claim 1, whereby said impression compound is reusable.

31. The impression tray, such as in particular a dental impression tray, of claim 1, whereby, after taking an impression, said impression compound reacts to applied agents, such as for example sprays or liquids, in order to bring about a data transfer and/or storage.

32. The impression tray, such as in particular a dental impression tray, of claim 1, whereby said impression compound is chosen such that its consistency is changed by supplied electrical energy.

33. The impression tray, such as in particular a dental impression tray, of claim 1, whereby said impression compound is of such a nature that it is a memory compound and accordingly has a memory effect, in that after activation it reverts to its original shape.

34. The impression tray, such as in particular a dental impression tray, of claim 1, further comprising a screen or display and/or input devices, such as in particular keys.

35. The impression tray, such as in particular a dental impression tray, of claim 1, whereby a film is provided, with which film said impression compound can be covered before an impression is taken, in order to prevent contact with saliva or mouth tissue/skin and/or to transmit data by deforming.

36. The impression tray, such as in particular a dental impression tray, of claim 1, whereby a camera is provided in or on said impression tray, in order to record an image of a patient or at least reference points and add it/them to jaw/tooth data determined.

37. A method for capturing structures, arrangements or shapes, such as preferably for capturing dental structures, arrangements or shapes in a mouth or in a human body, whereby a deformable impression compound is brought onto or into said structures, arrangements or shapes in particular, is introduced, into said mouth or body and a change of at least one physical property and/or variable of said impression compound is transmitted there in a spatially resolved manner directly to sensor devices when preparing an impression and is captured by said sensor devices and, furthermore, provided in a form that is suitable for electronic data processing.

38. The method of claim 37, whereby said sensor devices capture on said impression compound at least one of
change in radiation transparency or radiation absorption, in particular transparency to light or absorption of light,
change in electrical conductivity,
change in pressure, in particular by changes of conductivity as a result of said change in pressure,
deformation,
change in cross section or change in thickness,
change in electrical resistance,
change in the density or change in distribution of foreign atoms, in each case in particular by changes of electrical or optical conductivity as a result of said change in density or change in said distribution of foreign atoms.

39. The method of claim 37, whereby interface devices are coupled to said sensor devices on an output side, in order to pass on data generated by said sensor devices in a form suitable for electronic data processing, said interface devices preferably comprising a USB port.

40. The method of claim 39, whereby memory devices, in particular memory devices that can be decoupled, are also arranged downstream of said interface devices, preferably as a chip card or as a memory stick.

41. The method of claim 39, whereby connecting devices, in particular wireless connecting devices, are arranged downstream of said interface devices, preferably as Bluetooth®, infrared and/or radio devices.

42. The method of claim 37, whereby said impression compound, in accordance with said changes when it is pressed onto teeth or some other structure in said human body and a property thereof, passes on data to said sensor devices on a surface and on inner faces of said impression tray, such as in particular a dental impression tray.

43. The method of claim 37, whereby said data obtained are either stored directly in said impression tray, such as in particular in said dental impression tray, or are transmitted by cable, USB or radio to a central PC, where they are then used for further processing operations.

44. The method of claim 37, whereby said data are sent online directly to a dental laboratory for further processing.

45. The method of claim 37, whereby firstly a first impression is prepared with a first impression material, and then a second impression with, for example, additionally or alternatively a low-viscosity impression material, which when used in combination possibly in turn passes on information to said first impression material.

46. The method of claim 45, whereby said two impression materials are used with different impression trays.

47. The method of claim 37, whereby said impression compound is composed of three different impression materials that cannot be mixed with one another, of different colors or different transmission and/or reflection properties.

48. The method of claim 37, whereby said impression compound consists of a number of films placed one on top of the other, in particular of different colors.

49. The method of claim 37, whereby, to produce prostheses for parts of teeth as a whole, dental structures, individual teeth and parts of teeth, firstly an impression of an existing state before a treatment is prepared, and corresponding data are therewith determined, after that a treatment is performed, such as for example grinding down of a morbid tooth, an impression of a new state is once again prepared and corresponding data of said new state are determined, and then, by means of matching and/or difference methods, a prosthesis, such as for example a crown or bridge, with exact inner and outer shape and dimensions is produced from said data from said two impressions taken.

50. A device for capturing a three-dimensional structure of the human or animal body, in particular a tooth or set of teeth, which comprises the following:
a carrier for an impression compound,
an impression compound arranged on said carrier,
at least one lighting unit, which is designed for radiating light into said impression compound, and
at least one sensor unit, which is designed for detecting light emerging from said impression compound and generating spatially resolved raw data therefrom.

51. The device of claim 1, whereby said impression compound comprises at least one material which is selected from the group consisting of fluorescent materials, phosphorescent materials, light-diffusing materials and light-reflecting materials.

52. The device of claim 51, whereby said impression compound comprises at least one material which is selected from the group consisting of fluorescent materials and phosphorescent materials and whereby said at least one lighting unit is designed for emitting light of a wavelength which lies in the range of excitation of said fluorescent materials and/or said phosphorescent materials.

53. The device of claim 1, whereby said impression compound is optically transparent in at least one wavelength range.

54. The device of claim 53, whereby said lighting unit emits light of a wavelength which lies in a wavelength range of said optical transparency of said impression compound.

55. The device of claim 1, whereby said at least one lighting unit comprises a light source which is selected from the group consisting of LEDs, OLEDs, RGB-LEDs and laser LEDs.

56. The device of claim 1, whereby said at least one lighting unit is designed for projecting a pattern into said impression compound.

57. The device of claim 50, whereby said impression compound comprises a pattern which has been applied to it and/or incorporated in it.

58. The device of claim 50, whereby said at least one lighting unit is designed for emitting pulsed light.

59. The device of claim 58, whereby said raw data contain spatially resolved light transit time data.

60. The device of claim 50, whereby said raw data contain spatially resolved brightness data.

61. The device of claim 50, whereby said at least one sensor unit comprises a multiplicity of glass fibers and at least one optical sensor, whereby one end of said glass fibers is respectively aligned with said impression compound and whereby a second end of said glass fibers is respectively aligned with said at least one optical sensor.

62. Device of claim 61, whereby said at least one optical sensor is selected from the group consisting of CCD chips and CMOS chips.

63. The device of claim 50, further comprising a memory unit for storing said raw data generated by said at least one sensor unit.

64. The device of claim 50, further comprising a computing unit for generating image data from said raw data generated by said at least one sensor unit.

65. The device of claim 50, further comprising an interface for passing on said raw data generated by said at least one sensor unit or image data generated by a computing unit to a data processing unit.
